(19) United States
(12) Patent Application Publication
(10) Pub. No.: US 2012/0264703 A1
(43) Pub. Date: Oct. 18, 2012

(54) Methods And Compositions For The Treatment Of Anxiety Disorders, Including Post Traumatic Stress Disorder (PTSD) And Related Central Nervous System (CNS) Disorders.

(76) Inventors: Arifulla Khan, Sammamish, WA (US); John Frederick Reinhard, JR., Bellevue, WA (US)

(21) Appl. No.: 13/523,888

(22) Filed: Jun. 15, 2012

Related U.S. Application Data
(63) Continuation of application No. 13/308,444, filed on Nov. 30, 2011.
(60) Provisional application No. 61/417,909, filed on Nov. 30, 2010.
(60) Provisional application No. 61/542,129, filed on Sep. 30, 2011.

(51) Int. Cl. A61K 31/403; A61K 31/4045; A61K 31/18; A61K 31/517; A61K 31/138; A61K 31/417; A61K 31/4704; A61K 31/404; A61K 31/5377; A61K 31/167; A61K 31/165; A61K 31/17; A61K 31/216; A61K 31/352; A61K 31/166; A61K 31/343; A61K 31/53; A61K 31/55; A61K 31/19; A61K 31/7048; A61K 31/675; C07D 209/88; C07F 9/572; A61P 25/22; A61P 25/00; A61K 31/496

(52) U.S. Cl. 514/25; 514/411; 514/252.17; 514/415; 514/603; 514/266.24; 514/651; 514/401; 514/312; 514/652; 514/236.2; 514/629; 514/620; 514/597; 514/538; 514/456; 514/469; 514/242; 514/217; 514/557; 514/80; 514/77; 548/444; 548/414

(57) ABSTRACT

The present invention provides novel methods and formulations for treating anxiety disorders, including Post Traumatic Stress Disorder, in human subjects employing coordinate treatment using α and β blockers alone or in combination with additional psychotherapeutic medications to treat the anxiety disorder and reduce symptomology in treated subjects.

### RELATED APPLICATIONS

This application claims priority benefit of U.S. Provisional patent application Ser. No. 61/417,909, filed Nov. 30, 2010, and U.S. Provisional patent application Ser. No. 61/542,129, filed Sep. 30, 2011, the disclosure of which are incorporated herein in their entirety by reference.

### TECHNICAL FIELD

This application relates generally to compositions and methods for treating anxiety disorders. Specifically, this application relates to the use of α and/or β blockers in the treatment of anxiety disorders, particularly post-traumatic stress disorder.

### BACKGROUND OF THE INVENTION

Anxiety Disorders are among the most common mental health disorders, affecting about 40 million American adults age 18 years and older (about 18%) in a given year (Kessler et al. Arch. Gen. Psych 2005). They generally last at least six months and can get worse if not treated. While the cause is not clear, they are believed to have both biological, social and psychological components ranging from heredity, personality, life experiences including reactions to stress such as traumatic events, and brain chemistry such as low neurotransmitter levels and problems with amygdala functioning. Anxiety disorders can result in persistent and disabling psychological and physiological symptoms that interfere with the day to day life of an affected individual and include disorders such as acute stress disorder, panic disorder, generalized anxiety disorder, agoraphobia with or without panic disorder, specific phobia, social phobia, obsessive-compulsive disorder, separation anxiety disorder, and post-traumatic stress disorder.

Symptoms of anxiety disorders may vary depending on the disorder, but may include feelings of panic; persistent worry; doubt; dread; fear; uneasiness; uncontrollable, obsessive thoughts; repeated thoughts or flashbacks of traumatic experiences; mood instability; agitation; restlessness; dyspepsia; headaches; dyspnea; nightmares; ritualistic behaviors, such as repeated hand washing; insomnia; cold or sweaty hands and/or feet; shortness of breath; palpitations; an inability to be still and calm; intense startle reflex; dry mouth; numbness or tingling in the hands or feet; nausea; muscle tension; and/or dizziness.

Acute stress disorder is a result of a traumatic event in which the person experienced or witnessed an event that involved threatened or actual serious injury or death and responded with intense fear and helplessness. Symptoms include dissociative symptoms such as numbing, detachment, a reduction in awareness of the surroundings, derealization, or depersonalization, re-experiencing of the trauma, avoidance of associated stimuli, and significant anxiety, including irritability, poor concentration, difficulty sleeping, and restlessness. If left untreated, the condition may evolve into Post Traumatic Stress Disorder (PTSD).

Panic Disorder is characterized by sudden attacks of intense fear or anxiety, usually associated with numerous physical symptoms such as heart palpitations, rapid breathing or shortness of breath, blurred vision, dizziness, and racing thoughts. Generalized anxiety disorder is evidenced by general feelings of anxiety such as mild heart palpitations, dizziness, and excessive worry. Agoraphobia is the anxiety of being in places where escape might be difficult or embarrassing or in which help may not be available should a panic attack develop. Phobias result in extreme anxiety and/or fear associated with the object or situation of avoidance. Obsessive compulsive disorders are characterized by persistent, often irrational, and seemingly uncontrollable thoughts and actions which are used to neutralize the obsessions.

PTSD results from experiencing or witnessing a traumatic event that causes intense fear, helplessness or horror. It results in symptoms that fall into three types: re-experiencing the event, emotional numbing and avoidance and hyperarousal. Repetition of these overwhelming emotions can lead to a cascade of biological events including excessive release of epinephrine and norepinephrine which overpowers the autonomic response leading to clamminess, increased heart rate and breathing, increased blood flow to the muscles and decreased blood flow to the visceral organs. It is currently theorized that this response leads to deep imprinting on the locus coeruleus region of the brain and makes it over sensitized to any further threats (real or imaginary). (Diagnostic and Statistical Manual of Mental Disorders 4th edition (DSM-IV) published by the American Psychiatric Association (APA; Washington, D.C., 1994). PTSD is also believed to involve the serotonergic and endorphin system. (Holbrook et al, 2010). Experiments have consistently shown a serotonin deficit in “stressed” animals. Through multiple interconnections with the limbic system, serotonin has been found to mediate response to acute and chronic stress, conditioned fear, and flight or fight responses. Further, serotonin also modulates norepinephrine levels thereby leading to indirect effects on stress response through the adrenergic system.

Exposure to traumatic events is common with more than 50% of the US population experiencing one or more traumatic events in their lifetime. (Kessler et al., 1995) However, the rates of PTSD varies according to the population with a lifetime prevalence of approximately 5 to 12% of the population with women having twice the prevalence rate of men (Kessler et al., 1995) and certain segments of the population, such as combat soldiers having rates as high as 25%.

Anxiety disorders are generally treated with a combination of medication and psychotherapy. However, many of the currently prescribed medications merely keep anxiety disorders under control while psychotherapy is attempted, they do not actually treat the disorder. In the case of some disorders, very few medications have been approved. For example, only two medications, sertraline and paroxetine, have been approved by the FDA for treatment of Post Traumatic Stress Disorder. The medications currently used to treat anxiety disorders have unwanted characteristics and side effects including drug interactions, cardiovascular side effects, gastrointestinal side effects, sexual side effects, suicidal ideation and slow onset of action. Additionally, as shown in FIG. 1, medication for anxiety disorders elicits only a modest response in comparison to a placebo not only in terms of the primary rating scale, but in overall Clinical Global Impression as well. Even with treatment, residual symptoms and poor functioning continue to be a problem for those suffering from or at risk for anxiety disorders. There is therefore a need in the art for the discovery of additional treatments for anxiety disorders including PTSD.

### SUMMARY OF THE INVENTION

Provided herein are means for preventing, treating, ameliorating, alleviating and reducing signs and symptoms of anxiety disorders including Post Traumatic Stress Disorder (PTSD) in mammalian subjects including humans. These and other subjects are effectively treated by administering to the subject an effective amount of α and β blockers, a combined α and β blocker, or an α or β blocker. The α and β blockers may be administered alone or with the addition of one or more additional psychotherapeutic agents in an amount effective to prevent, treat, ameliorate, alleviate or reduce the anxiety disorder.

In one aspect of the invention, means are provided herein for treating or preventing symptoms of anxiety disorders, including PTSD, in female subjects. These women subjects are effectively treated by administering to the subjects an effective amount of α and β blockers or a combined α and β blocker. An exemplary α and β blocker as used herein is carvedilol.

In another aspect of the invention, means are provided herein for treating or preventing symptoms of anxiety disorders, including PTSD, in male and female subjects who are not military veterans. These subjects are effectively treated by administering to the subjects an effective amount of α and β blockers or a combined α and β blocker. An exemplary α and β blocker as used herein is carvedilol.

The invention further provides means for preventing or treating signs and symptoms of anxiety disorders, including PTSD, in mammalian subjects including humans, by administering an effective amount of a sustained release therapeutic agent. Such therapeutic agents include α and β blockers, a combined α and β blocker, or an α or β blocker. The sustained release therapeutic agent typically will provide an increased bioavailability of the agent compared to an immediate release dosage form of the agent.

In an additional aspect of the invention, prevention or treatment of an anxiety disorder in a human subject is provided by administering to the subject a sustained release dosage form of carvedilol. Administration of the sustained release form of carvedilol will achieve an increase in solubility and/or bioavailability compared to that obtained with an immediate released dosage form of carvedilol.

The invention further provides means of treating or preventing symptoms of PTSD in a human subject who is either suffering from or at risk for PTSD by administering carvedilol to the subject.

In another aspect of the invention, treatment or prevention of symptoms of an anxiety disorder, including PTSD, is provided by administering an effective amount of a prodrug of carvedilol to a human subject suffering from such symptoms. The prodrug of carvedilol will advantageously provide for increased solubility and/or bioavailability compared to the parent drug of carvedilol.

A further aspect of the invention is a carvedilol prodrug. Carvedilol is characterized by its limited solubility and bioavailability. The carvedilol prodrugs provided herein have increased solubility and/or bioavailability properties compared to the carvedilol parent drug.

Within additional aspects of the invention, combinatorial formulations are provided which employ α and β blockers and psychotherapeutic agents effective to prevent, treat, ameliorate, alleviate or reduce the anxiety disorder in the subject, including human subjects. Exemplary combinatorial formulations and coordinate treatment methods employ a psychotherapeutic agent including, but not limited to, drugs from the general classes of anti-depressant, mood-stabilizing, anxiolytic, anticonvulsant, antipsychotic, antiaddictive, appetite suppressant drugs, and opiate agonists. An exemplary α and β blocker as used herein is carvedilol.

In the coordinate administration methods of the invention, the α blocker, β blocker, or combined α and β blocker and the psychotherapeutic agent are administered concurrently or sequentially in any order to prevent or treat one or more symptoms of the targeted anxiety disorder, including PTSD. When administered simultaneously, the α blocker and the psychotherapeutic agent, β blocker and the psychotherapeutic agent, combined α and β blocker and the psychotherapeutic agent, or the combination of the α blocker, β blocker and the psychotherapeutic agent may be combined in a single composition or combined dosage form, or administered at the same time in separate dosage forms.

Anxiety disorders for treatment with the methods and compositions herein include, but are not limited to, acute stress disorder, panic disorder, generalized anxiety disorder, agoraphobia without panic disorder, specific phobia, social phobia, post-traumatic stress disorder, obsessive-compulsive disorder, and separation anxiety disorder.

Symptoms of anxiety disorders include, but are not limited to, feelings of panic, fear, and uneasiness; uncontrollable, obsessive thoughts; repeated thoughts or flashbacks of traumatic experiences; nightmares; ritualistic behaviors, such as repeated hand washing; insomnia; cold or sweaty hands and/or feet; shortness of breath; palpitations; an inability to be still and calm; dry mouth; numbness or tingling in the hands or feet; nausea; muscle tension; and/or dizziness. The methods, formulations and coordinate treatment methods of the invention are effective to modulate, alleviate, treat or prevent one or more symptom(s) of the anxiety disorder in a subject, including a mammalian subject. Such formulations and coordinate treatment methods may be administered prior to or shortly after a triggering event, or after development of symptoms of an anxiety disorder, including PTSD.

The foregoing and other objects, features, aspects and advantages of the present invention will become more apparent from the following sections.

### BRIEF DESCRIPTION OF THE DRAWINGS

FIG. 1 contains charts depicting (a) Mean percentage symptom improvement for placebo (□) and psychotherapeutics (▪) based on primary rating scale and (b) mean percentage symptom improvement of placebo (□) and psychotherapeutics (▪) based on clinical global impressions scale (CGI-s). (Kahn, et al. 2005)

FIG. 2 contains a chart depicting the change of score on the DTS for females and males treated with carvedilol and placebo.

FIG. 3 shows Kaplan-Meier Survival Analysis of Time to Remission for Women (Part A) and Men (Part B) treated with carvedilol or placebo.

FIG. 4 shows phosphocarvedilol.

FIG. 5 shows carvedilol phosphocholine.

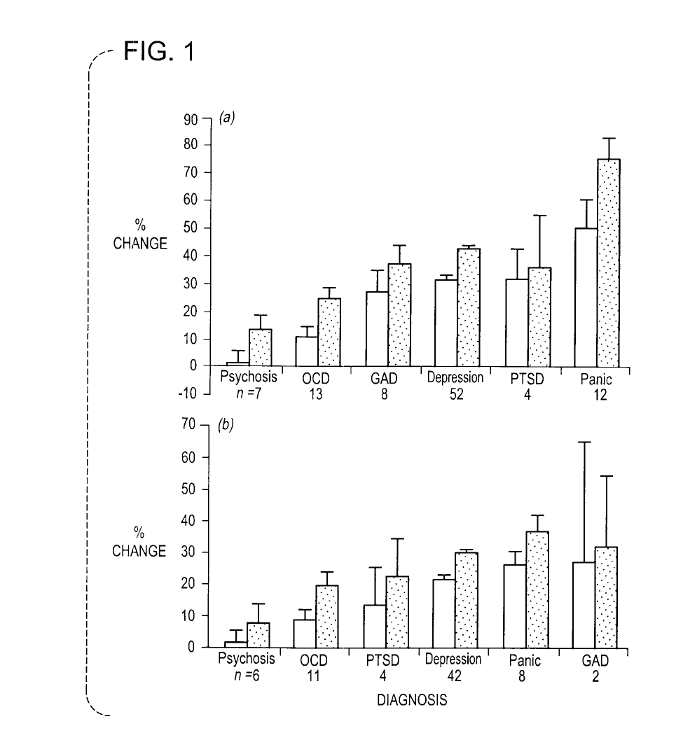

### DETAILED DESCRIPTION OF EXEMPLARY EMBODIMENTS

Anxiety Disorders categorize a large number of disorders where the primary feature is abnormal or inappropriate anxiety. These symptoms can occur without any recognizable stimulus or when the stimulus does not warrant such a reaction and can interfere with day to day living. The present invention provides novel methods and combined drug compositions, dosage forms, packages, and kits for preventing or treating anxiety disorders including Post Traumatic Stress Disorder (PTSD). The methods and compositions of the invention use α and/or β blockers or combined α and β blockers alone or in combination with other psychotherapeutic drugs to modulate, prevent, alleviate, ameliorate, reduce or treat the symptoms of anxiety disorders including PTSD. In some embodiments, administration of the compositions and methods of the present invention may prevent an anxiety disorder including PTSD from developing. In other embodiments, administration of the compositions and methods of the present invention may prevent recurrent episodes of an anxiety disorder including PTSD.

Subjects amenable to treatment according to the invention include mammalian subjects, including humans, suffering from or at risk for any of a variety of anxiety disorders including, but not limited to, acute stress disorder, panic disorder, generalized anxiety disorder, agoraphobia without panic disorder, specific phobia, social phobia, post-traumatic stress disorder, obsessive-compulsive disorder, and separation anxiety disorder. Within the methods of the invention an α and/or β blocker or combination α and β blocker is administered in an amount effective to treat a specified anxiety disorder alone or in combination with another psychotherapeutic drug including, but not limited to, drugs from the general classes of anti-depressant, mood-stabilizing, anxiolytic, anticonvulsant, antipsychotic, antiaddictive, appetite suppressant drugs and opiate agonists. (See, e.g., R J. Baldessarini in Goodman & Gilman's The Pharmacological Basis of Therapeutics, 9th Edition, Chapter 18, McGraw-Hill, 1996 for a review). In some embodiments an α blocker may be used, including an α-1 blocker. In other embodiments a β blocker may be used. In yet another embodiment a combination α and β blocker may be used. In a further embodiment an α and/or β blocker may be used in combination with one or more psychotherapeutic drugs.

In one embodiment, subjects amenable to treatment according to the invention include women suffering from or at risk for any of a variety of anxiety disorders including, but not limited to, acute stress disorder, panic disorder, generalized anxiety disorder, agoraphobia without panic disorder, specific phobia, social phobia, post-traumatic stress disorder, obsessive-compulsive disorder, and separation anxiety disorder. In a preferred embodiment, women subjects are amenable to treatment for PTSD, and may be treated with a combination of an α and β blocker. An exemplary single compound of an α and β blocker is carvedilol.

Symptoms of anxiety disorders vary and may include, but are not limited to, feelings of panic, fear, and uneasiness; uncontrollable, obsessive thoughts; repeated thoughts or flashbacks of traumatic experiences; nightmares; ritualistic behaviors, such as repeated hand washing; insomnia; cold or sweaty hands and/or feet; shortness of breath; palpitations; an inability to be still and calm; dry mouth; numbness or tingling in the hands or feet; nausea; muscle tension; and/or dizziness. The methods, formulations and coordinate treatment methods of the invention are effective to modulate, alleviate, treat or prevent one or more symptom(s) of the anxiety disorder in a subject, including a mammalian subject.

Exemplary formulations use an α and β blocker such as carvedilol alone or in combination with one or more psychotherapeutic drugs including, but not limited to, anti-depressants, mood-stabilizing, anxiolytic, anticonvulsant, antipsychotic, antiaddictive, appetite suppressant drugs and opiate agonists.

Within the coordinate administration methods of the invention, an α and β blocker such as carvedilol or separate α and/or β blockers are administered concurrently or sequentially with one or more psychotherapeutic drugs including, but not limited to, anti-depressants, mood-stabilizing, anxiolytic, anticonvulsant, antipsychotic, antiaddictive, appetite suppressant drugs, and opiate agonists to treat or prevent one or more symptoms of the targeted anxiety disorder, including PTSD. When administered simultaneously, the α blocker, β blocker and psychotherapeutic drug may be combined in a single composition or combined dosage form. Alternatively, the combinatorially effective α and β blockers such as carvedilol and psychotherapeutic agent(s) may be administered at the same time in separate dosage forms. When the coordinate administration is conducted simultaneously or sequentially, the α and β blockers and psychotherapeutic agent may each exert biological activities and therapeutic effects over different time periods, although a distinguishing aspect of all coordinate treatment methods of the invention is that treated subjects exhibit an alleviation or prevention of anxiety symptoms.

Dosing and therapeutic benefits of the α and/or β blocker coordinately administered with the psychotherapeutic drug will typically have similarly favorable therapeutic effects and comparable side effects as a therapeutic benefit and side effect profile achieved in control patients treated with the psychotherapeutic agent alone. However, in certain embodiments the dosage of the psychotherapeutic agent may be lowered and yet in combination with the α and/or β blocker, such as carvedilol, will still have comparable therapeutic benefits and similar side effects as a therapeutic benefit and side effect profile achieved in control patients treated with a higher dosage of the psychotherapeutic agent alone. Additionally, the α and/or β blocker, such as carvedilol, may also be present in lower or sub-therapeutic amounts yet in combination with the psychotherapeutic drug will still have comparable therapeutic benefits and similar side effects as a therapeutic benefit and side effect profile achieved in control patients treated with a higher dosage of the α and/or β blocker alone.

Within more detailed embodiments of the invention, the compositions and methods of the invention achieve substantial therapeutic benefit in terms of a clinical reduction in incidence, development, rate, recurrence, or severity of an anxiety disorder, particularly PTSD. In related embodiments, the compositions and methods of the invention measurably alleviate or prevent one or more symptoms of an anxiety disorder.

In more detailed embodiments of the invention, anxiety disorders, which disorder is typically defined as an extended period (e.g. at least six months, except in the instance of acute stress disorder) of excessive anxiety or worry with symptoms on most days of this period, is treated. In embodiments of the invention, anxiety disorders such as acute stress disorder, panic disorder, generalized anxiety disorder, agoraphobia, specific phobia, social phobia, obsessive-compulsive disorder, separation anxiety disorder, and post-traumatic stress disorder may be treated.

In one embodiment, the methods and compositions of the invention are employed to treat acute stress disorder is an anxiety disorder that can develop immediately following a traumatic event and lasts for no more than four weeks. Symptoms include re-experiencing the event, hyperarousal, avoidance, and dissociative symptoms such as feeling numb or attached. In some circumstances, it can evolve into PTSD.

In other detailed embodiments, the methods and compositions of the invention are employed to treat a panic disorder, defined as the presence of recurrent panic attacks followed by at least one month of persistent concern about having another panic attack. A “panic attack” is a discrete period in which there is a sudden onset of intense apprehension, fearfulness or terror. During a panic attack, the individual may experience a variety of symptoms including palpitations, sweating, trembling, shortness of breath, chest pain, nausea and dizziness. Panic disorder may occur with or without agoraphobia.

In a further embodiment, the methods and compositions of the invention are employed to treat generalized anxiety disorder which is characterized by 6 months or more of chronic, exaggerated worry and tension that is unfounded or much more severe than the normal anxiety most people experience

Alternate anxiety disorders amenable to treatment according to the invention include phobias, for example agoraphobia, specific phobias and social phobias. Agoraphobia is characterized by anxiety about being in places or situations from which escape might be difficult or embarrassing, for example on an airplane, or in which help may not be available in the event of a panic attack. Agoraphobia may occur without history of a panic attack. A “specific phobia” is characterized by clinically significant anxiety provoked by exposure to a specific feared object or situation. Specific phobias include subtypes such as animal type, cued by animals or insects; natural environment type, cued by objects in the natural environment, for example storms, heights or water; blood-injection-injury type, cued by the sight of blood or an injury or by seeing or receiving an injection or other invasive medical procedure; situational type, cued by a specific situation such as public transportation, tunnels, bridges, elevators, flying, driving or enclosed spaces; and other type where fear is cued by other stimuli. A “social phobia” is characterized by clinically significant anxiety provoked by exposure to certain types of social or performance circumstances. Social phobia may also be referred to as social anxiety disorder.

The methods and compositions of the invention may also be used to treat PTSD. PTSD is characterized by the development of symptoms following exposure to an extreme traumatic stressor. The traumatic stressor must involve direct personal experience of an event that involves actual or threatened death or serious injury, or other threat to one's physical integrity; witnessing an event that involves death, injury, or a threat to the physical integrity of another person; or learning about unexpected or violent death, serious harm, or threat of death or injury experienced by a family member or other close associate.

Exemplary traumatic events include, but are not limited to military combat, violent personal assault, being kidnapped, being taken hostage, terrorist attack, torture, incarceration as a prisoner of war or in a concentration camp, natural or manmade disasters, severe automobile accidents, or being diagnosed with a life-threatening illness; observing the serious injury or unnatural death of another person due to violent assault, accident, war, or disaster or unexpectedly witnessing a dead body or body parts; learning about violent personal assault, serious accident, or serious injury experienced by a family member or a close friend; learning about the sudden, unexpected death of a family member or a close friend; or learning that one's child has a life-threatening disease.

Symptoms include, but are not limited to, persistent re-experiencing of the traumatic event, persistent avoidance of stimuli associated with the trauma and numbing of general responsiveness, and persistent symptoms of hyperarousal for more than 1 month. Additionally, the symptoms must cause clinically significant distress or impairment in social, occupational, or other important areas of functioning. The traumatic event can be re-experienced in various ways. Commonly the person has recurrent and intrusive recollections of the event or recurrent distressing dreams during which the event is replayed. In rare instances, the person experiences dissociative states that last from a few seconds to several hours, or even days, during which components of the event are relived and the person behaves as though experiencing the event at that moment. Intense psychological distress or physiological reactivity often occurs when the person is exposed to triggering events that resemble or symbolize an aspect of the traumatic event. Avoidance/numbing may include efforts to avoid thoughts, feelings, conversations about the traumatic event, activities, situations, or people who arouse recollections of it. In some instances, avoidances may include amnesia regarding the event. Numbing may manifest as markedly diminished interest or participation in previously enjoyed activities, feeling detached or estranged from other people, or of having markedly reduced ability to feel emotions. Hyperarousal may manifest as difficulty falling or staying asleep that may be due to recurrent nightmares during which the traumatic event is relived, hypervigilance, and exaggerated startle response. Some individuals report irritability or outbursts of anger or difficulty concentrating or completing tasks. (See 309.81 DSM-IV Criteria for Post-traumatic Stress Disorder).

It has been theorized that there may be multiple subtypes of patients with PTSD, including those with sensitized noradrenergic systems, sensitized serotonergic systems (Southwick et al., 1997), and endorphin system involvement (Holbrook et al., 2010). Investigations into PTSD have found alterations in the hypothalamic-pituitary-adrenocortical axis and in catecholaminergic and serotonergic systems (Marshall et al., 2001). In certain embodiments, the psychological response triggers one or more physical responses including, but not limited to, abnormal respiration, abnormal cardiac rhythm, abnormal blood pressure, or abnormal sensory processing.

In particular, the methods and compositions of the invention may be used to treat PTSD in women, or men and women who are not military veterans. As detailed in Example 4 herein, a clinical trial was performed in which men and women subjects suffering from PTSD were treated with the α and β blocker compound carvedilol or placebo. Surprisingly, carvedilol was efficacious in treating women suffering from PTSD, but not men. The therapeutic effect of carvedilol in women was large, whereas placebo seemed to have a larger effect than carvedilol in men. Furthermore, the results indicate that women suffering from PTSD associated with personal assault (physical and sexual) may be specifically sensitive to treatment with carvedilol, since the greatest treatment effect was observed in this subgroup of women. Females suffering from PTSD appear to undergo physiological and psychological changes that are more likely to be treated by carvedilol than those changes experienced by men.

However, further analysis of the clinical trial detailed in Example 4 indicated that PTSD can be effectively treated in both male and female subjects who were not military veterans. These results indicate that the overall findings that carvedilol was effective in treating PTSD in females but not males may have been skewed by the inclusion of military veterans. In the overall study, a large percentage of the males treated with either carvedilol or placebo were military veterans, while only one of the women (who received placebo) was a military veteran. Male PTSD subjects who are military veterans may have a subset of physiological and psychological exposure that renders carvedilol ineffective in that sub-population. Men suffering from PTSD who are not military veterans and who have not undergone military training may possess a physiological and psychological make-up that allows treatment with carvedilol to be effective.

In the cases of anxiety disorder with external stimuli such as acute stress disorder or PTSD, the compositions and methods of the present invention may be used prophylactically. For example, the compositions of the present invention may be administered prior to exposure to a traumatic event or shortly after a traumatic event as well as after development of symptoms.

Administration of a α and/or β blocker or the coordinate treatment method or combinatorial drug composition of the invention to suitable subjects (e.g., qualified subjects suffering from an anxiety disorder or at increased risk for developing an anxiety disorder) will yield a reduction in one or more target symptom(s) associated with the selected anxiety disorder or development of the anxiety disorder by at least 10%, 20%, 30%, 50% or greater, up to a 75-90%, or 95% or greater, compared to placebo-treated or other suitable control subjects. Comparable levels of efficacy are contemplated for the entire range of anxiety disorders described herein, including all contemplated neurological and psychiatric disorders, and related conditions and symptoms, for treatment or prevention using the compositions and methods of the invention. These values for efficacy may be determined by comparing accepted therapeutic indices or clinical values for particular test and control individuals over a course of treatment/study, or more typically by comparing accepted therapeutic indices or clinical values between test and control groups of individuals using standard human clinical trial design and implementation.

In one embodiment, administration of carvedilol to women suffering from PTSD will yield a reduction in one or more target symptom(s) associated with PTSD by at least 10%, 20%, 30%, 50% or greater, up to a 75-90%, or 95% or greater, compared to placebo-treated women or other suitable control women subjects.

As used herein, the terms “prevention” and “preventing,” when referring to an anxiety disorder or symptom, refers to a reduction in the risk or likelihood that a mammalian subject will develop said disorder, symptom, condition, or indicator after treatment according to the invention, or a reduction in the risk or likelihood that a mammalian subject will exhibit a recurrence of said disorder, symptom, condition, or indicator once a subject has been treated according to the invention and cured or restored to a normal state (e.g., placed in remission from a targeted anxiety disorder). As used herein, the terms “treatment” or “treating,” when referring to anxiety disorders, particularly post traumatic stress disorder (PTSD), refers to inhibiting or reducing the progression, nature, or severity of the subject condition or delaying the onset of the condition.

An “effective amount,” “therapeutic amount,” “therapeutically effective amount,” or “effective dose” of an α and/or β blocker and/or a psychotherapeutic agent as used herein means an effective amount or dose of the active compound as described herein sufficient to elicit a desired pharmacological or therapeutic effect in a human subject. In the case of anti-anxiety therapeutic agents, these terms most often refer to a measurable, statistically significant reduction in an occurrence, frequency, or severity of one or more symptom(s) of a specified anxiety disorder, including any combination of neurological and/or psychological symptoms, diseases, or conditions, associated with or caused by the targeted anxiety disorder.

Therapeutic efficacy can alternatively be demonstrated by a decrease in the frequency or severity of symptoms associated with the treated condition or disorder, or by altering the nature, occurrence, recurrence, or duration of symptoms associated with the treated condition or disorder. Therapeutic efficacy with the treated condition or disorder, or by altering the nature, recurrence, or duration of symptoms associated with the treated condition or disorder In this context, “effective amounts,” “therapeutic amounts,” “therapeutically effective amounts,” and “effective doses” of psychotherapeutic drugs and α and/or β blockers within the invention can be readily determined by ordinarily skilled artisans following the teachings of this disclosure and employing tools and methods generally known in the art, often based on routine clinical or patient-specific factors. In some embodiments, therapeutic efficacy will be determined prophylactically in that fewer or no subjects will develop the anxiety disorder in comparison to the related population. For example, in the case of combat soldiers, fewer than 25% of the treated population would develop symptoms of PTSD.

Efficacy of the coordinate treatment methods and drug compositions of the invention will often be determined by use of conventional patient surveys or clinical scales to measure clinical indices of anxiety disorders including PTSD in subjects. The methods and compositions of the invention will yield a reduction in one or more scores or selected values generated from such surveys or scales completed by test subjects (indicating for example an incidence or severity of a selected anxiety disorder), by at least 10%, 20%, 30%, 50% or greater, up to a 75-90%, or 95% compared to correlative scores or values observed for control subjects treated with placebo or other suitable control treatment. In at risk populations, the methods and compositions of the invention will yield a stable or minimally variable change in one or more scores or selected values generated from such surveys or scales completed by test subjects. More detailed data regarding efficacy of the methods and compositions of the invention can be determined using alternative clinical trial designs.

Useful patient surveys and clinical scales for comparative measurement of clinical indices of anxiety disorders in subjects treated using the methods and compositions of the invention can include any of a variety of widely used and well known surveys and clinical scales.

Among these useful tools are the Mini International Neuropsychiatric Interview© (MINI) (Sheehan et al., 1998); Clinical Global Impression scale (CGI) (Guy, W., ECDEU Assessment Manual for Psychopharmacology, DHEW Publication No. (ADM) 76-338, rev. 1976); HAM-A rating scale for anxiety (Hamilton, 1959); Clinician-Administered Post-traumatic Stress Disorder Scale (CAPS) (Weathers et al., 1999); Clinician-Administered PTSD Scale Part 2 (CAPS-2) (Blake et al., 1995); Clinician-Administered PTSD Scale for Children and Adolescents (CAPS-CA) (Nader et al., 1996); Impact of Event Scale (IES) (Horowitz et al. 1979); Impact of Event Scale-Revised (IES-R) (Weiss et al. 1996); Clinical Global Impression Severity of Illness (CGI-5) (Guy, 1976); Clinical Global Impression Improvement (CGI-I) (Guy, et al. 1976); Duke Global Rating for PTSD scale (DGRP) (Davidson et al., 1998); Duke Global Rating for PTSD scale Improvement (DGRP-I); Structured Interview for PTSD (SI-PTSD) (Davidson, et al. 1990); PTSD Interview (PTSD-1) (Watson et al., 1991); PTSD Symptom Scale (PSS-I) (Loa et al., 2006); Beck Depression Inventory (BDI) (Beck, 2006); Revised Hamilton Rating Scale for Depression (RHRSD) (Warren, 1994); Major Depressive Inventory (MDI) (Olsen et al. 2003); and Children's Depression Index (CDI) (Kovacs, et al. 1981).

Any of these scales, alone or in combination, can be effectively employed to determine efficacy of the methods and compositions of the invention. Additionally, a variety of other scales and methods for assessing comparative anxiety disorder symptoms or status, are widely used and well known in the art for use within the invention. Other exemplary scales for assessing efficacy of the invention include, for example, the Hamilton Depression Rating Scale© (HDRS) (Hamilton, M., J. Neurol. Neurosurg. Psychiatr. 23:56-62, 1960; Hamilton, M., Br. J. Soc. Clin. Psychol. 6:278-296, 1967); Montgomery-Asberg Depression Rating Scale© (MADRS) (Montgomery and Asberg, 1979); Beck Scale for Suicide Ideation® (BSS) (Beck and Steer, 1991 Columbia-Suicide Severity Rating Scale© (C-SSRS) or Columbia Classification Algorithm of Suicide Assessment© (C CASA) (Posner, K, et al., 2007); Sheehan-Suicidality Tracking Scale© (S—SST) (Coric et al., 2009); Beck Hopelessness Scale© (BHS) (Beck, Steer, 1988); Geriatric Depression Scale (GDS) (Yesavage, J. A. et al., J. Psychiatr. Res. 17:37-49, 1983). HAM-D scale for depression (Hamilton, 1960); the Yale-Brown Obsessive Compulsive Scale (YBOCS) (Goodman et al., 1989); The Positive and Negative Syndrome Scale (PANSS) for schizophrenia (Kay et al., 1987); the YMRS rating scale for mania (Young et al., 1978); the Liebowitz Social Anxiety Scale (Liebowitz).

In certain embodiments of the invention, efficacy of the methods and compositions provided herein is determined by use of the MINI scale, The MINI (e.g., version 6.0, January 2009, Sheenan et al., 1998), a clinician-rated diagnostic assessment. In the exemplary protocol described in Example 4 below, the MINI test is administered at the first, screening visit and will be considered a source document. The MINI is administered by a psychiatrist, psychologist or master's level clinician with a minimum of 2 years experience in the diagnosis of mental illness. Also for use in demonstrating efficacy of the invention is the Clinician Administered PTSD Scale (CAPS) (Westhers, F D et al, 1999) which was developed at the National Center for PTSD and consists of 30 carefully worded interview questions that target DSM-IV criteria for PTSD without leading the respondent. The Davidson Trauma Scale (DTS) (Davidson, J R et al, 1999) can also be employed, alone or in combination with any of the other scales or like tools known in the art. Likewise, the Insomnia Severity Index (ISI) (Bastien, C H et al, 2001) which evaluates the severity of sleep onset and maintenance difficulties, satisfaction with current sleep pattern, interference with daily functioning, appearance of impairment attributed to the sleep problem, and the degree of concern caused by insomnia and the Clinical Global Impression (CGI) (Guy, 1976) which evaluates the severity of the illness and illness improvement.

The methods and compositions of the invention will yield a reduction in one or more scores or values generated from these clinical surveys (using any single scale or survey, or any combination of one or more of the surveys described above) by at least 10%, 20%, 30%, 50% or greater, up to a 75-90%, or 95% compared to correlative scores or values observed for control subjects treated with placebo or other suitable control treatment. In prophylactic treatment, the methods and compositions of the invention will yield a stabilization or diminished change in the scores or values generated from these clinical surveys. For example, the Clinical Global Impression (CGI) scale is a 7-point, clinician rated scale to determine severity, improvement and response to treatment for selected anxiety disorders, including PTSD. The CGI severity of illness scale uses a range of responses from 1 to 7, with 1 being “normal” and 7 “amongst the most severely ill patients” (Guy, 1976). A “responder” according to this measuring tool is defined as being “Much Improved” or “Very Much Improved”, having a CGI score of at least 2. Thus in one alternate expression of efficacy of the invention, a frequency of normal to moderately symptomatic CGI scores, for example scores of 1, 2, 3, or 4, will occur more often in subjects treated according to the invention, by a factor of at least 10%, 20%, 30%, 50% or greater, up to a 75-90%, or 95% compared to a frequency of the same normal to moderately symptomatic scores or values observed for control subjects completing the CGI following administration of placebo. Yet another exemplary expression of efficacy for the coordinate treatment methods and combinatorial compositions of the invention involves The Davidson Trauma Scale which assesses the 17 DSM-IV symptoms of PTSD. Items are rated on 3-point frequency (0=“not at all” to 4=“every day”) and severity scales (0=“not at all distressing” to 4=“extremely distressing”). Respondents are asked to identify the trauma that is most disturbing to them and to rate, in the past week, how much trouble they have had with each symptom. The DTS yields a frequency score (ranging from 0 to 68), severity score (ranging from 0 to 68), and total score (ranging from 0 to 136). It can be used to make a preliminary determination about whether the symptoms meet DSM criteria for PTSD. Scores can also be calculated for each of the 3 PTSD symptom clusters. Following treatment according to the invention, a frequency of low Davidson Trauma Scale scores, will occur more often by at least 10%, 20%, 30%, 50% or greater, up to a 75-90%, or 95% compared to a frequency of the same low Davidson Trauma Scale scores observed in control subjects.

Additionally, anxiety disorders are among the few mental disorders for which animal models are available. Researchers can reproduce symptoms of human anxiety in test animals by manipulating physical or psychosocial stressors. These animal models provide additional means for determining effective coordinate treatment methods and combinatorial formulations of the invention. One of ordinary skill in the art will appreciate there are a number of animal models available for assessing anti-anxiety effects of compounds and methods of the invention. Two pharmacologically validated animal models of anxiety are the elevated zero maze test, and the isolation-induced ultrasonic emission test (Bickerdike, M. J. et al., Eur. J. Pharmacol., 271, 403 411 (1994); Shepherd, J. K. et al., Psychopharmacology, 116, 56 64 (1994)). Clinically used anxiolytic drugs, such as the benzodiazepines, increase the proportion of time spent in, and the number of entries made into, the open compartments. A second test for an anti-anxiety compound is the ultrasonic vocalization emission model, which measures the number of stress-induced vocalizations emitted by rat pups removed from their nest (Insel, T. R. et al., Pharmacol. Biochem. Behav., 24, 1263 1267 (1986); Miczek, K. A. et al., Psychopharmacology, 121, 38 56 (1995); Winslow, J. T. et al., Biol. Psychiatry, 15, 745 757 (1991). Additionally, animal models specific for PTSD may be used for measuring anxiolytic efficacy, for example, exposure to inescapable electric shock (e.g., Maier et al. 2001), high and low anxiety behavior rats (Muigg et al. 2008), single prolonged stress rats (Zhe et al., 2008), and the predator exposure model (Zoladz et al., 2008).

Compounds and methods of the invention can be administered to an animal to determine whether they affect anxiety or anxiety simulating behaviors of the animal. In some embodiments, the animals may be administered the compositions of the present invention prior to exposure to the anxiety inducing stimuli. In other embodiments, the compositions of the present invention may be administered just after conditioning by the anxiety inducing stimuli. In yet another embodiment, the compositions of the present invention may be administered after establishment of anxiety or anxiety-simulating behaviors.

Compositions of the invention may include α blockers, β blockers, combination α and β blockers. A preferred combination α and β blocker is carvedilol. Carvedilol, or (±) 1-(9H-carbazol-4-yloxy)-3-[[2(2-methoxyphenoxy)ethyl]amino]-2-propanol, (CAS Registry No. 72956-09-3), has a chiral center and can exist either as individual stereoisomers or in racemic form. The nonselective β-adrenergic activity of carvedilol is present in the S(−) enantiomer and the α-blocking activity is present in both the R(+) and S(−) enantiomers at equal potency (U.S. Pat. No. 7,056,942). Both the racemate and stereoisomers may be obtained according to procedures well known in the art (EP B 0127 099). As used herein, “carvedilol” refers to the racemate, which is the preferred embodiment of the present invention. However, the isomers of carvedilol may also be used in the present invention.

Carvedilol can be isolated as different polymorphs, each of which may be incorporated into the compositions of the present invention either individually or in combinations thereof. Two polymorphs of carvedilol are designated Form I and Form II, which are monotropic and are distinguishable by their infrared, Raman, and X-ray powder diffraction spectra, as described in international patent application publication No. WO 99/05105, incorporated herein by reference. Additional carvedilol polymorphs include Forms III, IV, V, which are distinguishable by their X-ray diffraction patterns, as described in U.S. Pat. No. 7,056,942, which is incorporated herein by reference.

In addition to carvedilol, compositions of the present invention may include prodrugs of carvedilol, which are metabolized or converted to yield active carvedilol. Carvedilol has low water solubility. Prodrugs and other modified forms of carvedilol may be employed to increase its solubility and commensurately, its oral bioavailability. The low oral bioavailability of carvedilol may potentially lead to differences in the blood levels of carvedilol following oral administration, thereby potentially rendering the effective dose as too high or too low in various patients. Increasing the bioavailability of carvedilol may therefore result in reduced inter-subject variability in drug exposure and a more normalized therapeutic delivery and effectiveness.

Prodrugs include compounds of the invention, for example carvedilol or carvedilol derivatives, wherein one or more appropriate active groups or chemically modifiable moieties of the parent drug have been modified to improve solubility (in physiological solutions, including blood and other tissues and compartments of mammalian subjects), bioavailability, half-life, and/or pharmacological activity in vivo, and generally the subject modification may be reversed upon administration to a mammalian, e.g., human, subject. Reversion is usually achieved by an enzyme naturally present in the subject, such as an endogenous phosphatase, though it is possible for a second agent to be administered together with a prodrug in order to mediate the prodrug reversion to a more active form, most often the parental drug form, in vivo.

The term “prodrug”, as used herein typically refers to a derivative of an active compound or drug that requires a transformation under the conditions of use, such as within the body, to release the active drug. Prodrugs are frequently, but not necessarily, pharmacologically inactive until converted into the active drug. Prodrugs are typically obtained by masking a functional group in the drug believed to be in part required for activity with a progroup to form a promoiety which undergoes a transformation, such as cleavage, under the specified conditions of use to release the functional group, and hence the active drug. The cleavage of the promoiety can proceed spontaneously, such as by way of a hydrolysis reaction, or it can be catalyzed or induced by another agent, such as by an enzyme, by light, by acid, or by a change of or exposure to a physical or environmental parameter, such as a change of temperature. The agent can be endogenous to the conditions of use, such as an enzyme present in the cells to which the prodrug is administered or the acidic conditions of the stomach or it can be supplied exogenously.

A wide variety of progroups, as well as the resultant promoieties, suitable for masking functional groups in active drugs, e.g., carvedilol, to yield prodrugs, are useful targets. For example, a hydroxyl functional group can be masked as a sulfonate, ester or carbonate promoiety, which can be hydrolyzed in vivo to revert the hydroxyl group. An amino functional group can be masked as an amide, carbamate, imine, urea, phosphenyl, phosphoryl or sulfenyl promoiety, which can be hydrolyzed in vivo to revert the amino group. A carboxyl group can be masked as an ester (e.g., silyl esters and thioesters), amide or hydrazide promoiety, which can be hydrolyzed in vivo to revert the carboxyl group. Other examples of suitable progroups and their respective promoieties will be apparent to those of skill in the art.

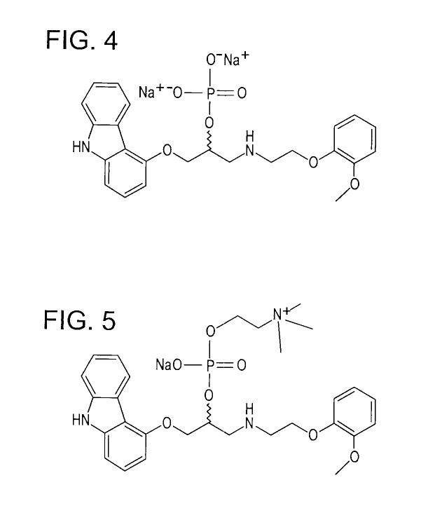

Carvedilol is a highly lipophilic molecule with an estimated octanol water partition coefficient (logP) of 3.585. Within the methods and compositions of the invention it has been discovered that the solubility, bioavailability, half-life, and/or pharmacological activity in vivo of carvedilol can be increased using novel carvedilol prodrug derivatives, including but not limited to phosphoester prodrugs of carvedilol. Thus in exemplary prodrug modifications useful within the invention for carvedilol include ester modifications, the parent drug is modified to an ester derivative prodrug form, and reversion may be carried out be an esterase, etc. Exemplary phosphoester carvedilol prodrugs described herein can be metabolized through hydrolysis by alkaline phosphatase to yield the parent carvedilol molecule or other active derivative or analog form of the drug in vivo. An example of such a carvedilol prodrug is a phospho ester disodium salt as shown in FIG. 4. Addition of a phosphate as described in this exemplary prodrug conversion increases solubility, bioavailability and pharmacological activity of the molecule, which has a calculated log P of 2.918 (determined using chemoinformatic software from www.molinspiration.com). This phospho ester disodium salt of carvedilol may be prepared by dissolving the free base of carvedilol in dimethylformamide (DMF) and adding a 3-fold molar equivalent of phosphorus oxychloride (POCl₃), as described in Example 7, or using other methods and reagents known in the art. The phosphate can be isolated using, for example, anion exchange chromatography, and the salt formed by exposure to 2 equivalents of base, typically sodium hydroxide. Other salts may also be produced according to the teachings herein using methods and materials known in the art, including, without limitation, magnesium and calcium salts.

Additional prodrug derivatives of carvedilol may also be prepared from a carvedilol phosphate, such as that shown in FIG. 4. Such derivatives include, but are not limited to, phosphocholine derivatives. Since phosphocholine is a naturally occurring component of lipid membranes, a phosphocholine ester derivative of carvedilol is likely to undergo hydrolysis during its passage across cell membranes, and its passage may be potentially aided by phospholipid transport proteins. An example of such a phosphocholine ester of carvedilol is shown in FIG. 5. The phosphocholine ester should have dramatically increased water solubility as it has a calculated LogP of −0.753. Additionally, the phosphocholine ester may render the molecule a transport substrate that should undergo hydrolysis by ubiquitous phospholipases. Additional prodrug ester derivatives of carvedilol may include sulfate ester derivatives, which will yield advantageous hydrophilicity/solubility properties for certain applications of the methods and compositions of the invention.

Additional prodrug ester derivatives of carvedilol and other active compounds for use within the invention are provided herein, which may be produced by additional prodrug ester modifications directed to, for example, the following alternative active groups or moieties of the subject parent compound, alkyl, aryl, arylalkyl, alkoxy, alkoxyl, etc., modified to any of a range of contemplated ester prodrug forms convertible in vivo by reversion to the parent compound, or otherwise converted to yield a modified, active drug compound in vivo. In accordance with this aspect of the invention, additional convertible ester prodrug derivatives of carvedilol and other therapeutic compounds for use within the invention will include a range of physiologically hydrolyzable esters, including alkylbenzyl, methoxybenzyl, indanyl, phthalyl, methoxymethyl, alkanoyloxy-alkyl, acetoxymethyl, alkoxycarbonyloxy-alkyl, glycyloxymethyl, phenylglycyloxymethyl, and other physiologically hydrolyzable esters that may be prepared using conventional techniques known in the art.

Useful methods and materials to produce additional prodrug derivatives of carvedilol and other therapeutic compounds for use within the invention may be found, for example in Design of Prodrugs, edited by H. Bundgaard, (Elsevier, 1985) and Methods in Enzymology, 112:309-396, edited by K. Widder, et al. (Academic Press, 1985); A Textbook of Drug Design and Development, edited by Krosgaard-Larsen and H. Bundgaard, Chapter 5, “Design and Application of Prodrugs,” by H. Bundgaard, pp. 113-191 (1991); and H. Bundgaard, Advanced Drug Delivery Reviews, 8:1-38 (1992), each incorporated herein by reference.

The solubility, bioavailability and/or pharmacological activity of carvedilol can also be increased by modifying the compound to yield salt and other forms of carvedilol that have an increased solubility compared to the parent compound. Such modifications include modification with an acid ester. For example, citric acid may be used to generate a citric acid ester or citrate ester of carvedilol. Pharmaceutically acceptable salts of carvedilol or other active therapeutic compounds for use within the methods and compositions of the invention include salts formed with inorganic and organic bases. Such salts, including ammonium salts; alkali metal salts, such as lithium, sodium and potassium salts; alkaline earth metal salts, such as calcium and magnesium salts; salts with organic bases, such as amine like salts (e.g., dicyclohexylamine salt, benzathine, N-methyl-D-glucamine, and hydrabamine salts); and salts with amino acids like arginine, lysine and the like; and zwitterions, the so-called “inner salts”. Pharmaceutically acceptable salts in this context also include acid addition salts, for example salts formed with strong inorganic acids, such as mineral acids, for example sulfuric acid, phosphoric acid or a hydrohalic acid such as HCl or HBr; with strong organic carboxylic acids, such as alkanecarboxylic acids of 1 to 4 carbon atoms which are unsubstituted or substituted, for example, by halogen, for example acetic acid, such as saturated or unsaturated dicarboxylic acids, for example oxalic, malonic, succinic, maleic, fumaric, phthalic or terephthalic acid, such as hydroxycarboxylic acids, for example ascorbic, glycolic, lactic, malic, tartaric or citric acid, such as amino acids, (for example aspartic or glutamic acid or lysine or arginine), or benzoic acid, or with organic sulfonic acids, such as (C.sub.1-4)alkyl or arylsulfonic acids which are unsubstituted or substituted, for example by halogen, for example methanesulfonic acid or p-toluenesulfonic acid.

Prodrug modifications, formation of pharmaceutically acceptable salts, and other modifications of carvedilol and other therapeutic compounds described herein yield improved solubility, bioavailability, and/or pharmacological efficacy, e.g., of carvedilol, of at least 10%, 20%, 20-30%, up to 30-50%, 50-70%, 100%, 200%, or greater, e.g., as compared to an equivalent dose of the unmodified parent drug, such as carvedilol. The improved solubility, bioavailability, and/or pharmacological efficacy of carvedilol prodrugs and salts and other modified therapeutic compounds within the invention can be readily determined using standard dissolution assays, pharmacokinetic studies, and/or in vitro and in vivo models of pharmacological activities, including in more detailed aspects known useful physiological models of anxiety disorders, such as PTSD. The increase in bioavailability and/or pharmacological activity of ester and other carvedilol prodrugs, carvedilol salts, and other modified therapeutic compounds provided herein, as determined e.g., using physiological models, such as animal models and/or human clinical studies, will be at least 10%, 20%, or 20-30%, up to 30-50%, 50-70%, 100%, 200% or greater compared to an equal dose of unmodified carvedilol parent drug.

Compositions of the invention may include α blockers, β blockers, combination α and β blockers and one or more psychotherapeutic agents or any combination thereof. α blockers contemplated for use in the present invention may include, but are not limited to, doxazosin, silodosin, prazosin, tamsulosin, alfuzosin, terazosin or trimazosin. Other α blockers include phenoxybenzamine and phentolamine. β blockers for use within the compositions and methods of the present invention include, but are not limited to alpreolol, bucindolol, carteolol, nadolol, penbutolol, pindolol, propanolol, timolol, acebutolol, atenolol, betaxonnlol, bisoprolol, celiprolol, esmolol, metoprolol, nebivolol, butazamine, ICI-118,551, or SR 59230A. Compositions and formulations of the present invention comprise these blockers alone or in combination with each other and/or with an additional psychotherapeutic agent. Additionally, combination α and β blockers, including carvedilol and labetalol, may be used alone or in combination with additional therapeutic compounds or drug agents, including adjunctive psychotherapeutic agents, within the methods and compositions of the invention. In exemplary embodiments of the invention, carvedilol is employed as both the α blocker and β blocker. Within this aspect of the invention, racemic carvedilol is preferred because the racemic form of the drug provides dual α blocker and β blocker functions, which are unequally distributed among the constituent enantiomeric forms of the drug.

In certain embodiments of the invention, an adjunctive psychotherapeutic agent is employed in combination with the α blocker and β blocker, for example in combination with carvedilol, a combined α and β blocking drug. The psychotherapeutic agent may be selected from known anti-depressant drugs, for example, any species within the broad families of tri-cyclic anti-depressants (TCAs) including, but not limited to, amitriptyline, imipramine, or desipramine; specific monoamine reuptake inhibitors, e.g., selective serotonin reuptake inhibitors (SSRIs) including, but not limited to, fluoxetine, fluvoxamine, sertraline and paroxetine, selective norepinephrine reuptake inhibitors, selective dopamine reuptake inhibitors, multiple monoamine reuptake inhibitors, monoamine oxidase inhibitors (MAOIs), noradrenaline reuptake inhibitors (NRIs), multiple monoamine reuptake inhibitors, e.g., that inhibit both serotonin and norepinephrine reuptake (SNRIs) including, but not limited to, venlafaxine and duloxetine, and indeterminate (atypical) anti-depressants are useful within this aspect of the invention. The psychotherapeutic agent may additionally include atypical antipsychotics including, but not limited to, Aripiprazole, Ziprasidone, Risperidone, Quetiepine, or Olanzapine or anticonvulsants including but not limited to lamotrigine, carbamazepine, oxcarbazepine, valproate, levetriacetam, and topiramate.

Within exemplary embodiments of the invention one or more of the anti-depressant drugs identified in Table 1 below is coordinately administered or combinatorially formulated with an α and/or β blocker to treat an anxiety disorder (e.g., depression or anxiety), including but not limited to PTSD. Single drugs, or multiple drugs from one or more of the indicated drug classes, may be co-administered, simultaneously or sequentially, with the α and/or β blocker, which may be combinatorially formulated with the psychotherapeutic therapeutic drug or provided in a separate dosage form.

TABLE 1
EXEMPLARY ANTI-DEPRESSANT DRUGS FOR COORDINATE
ADMINISTRATION WITH A AND/OR B BLOCKER

SSRIs
Celexa ® (citalopram)
Lexapro ® (excitalopram oxalate)
Luvox ® (fluvoxamine)
Paxil ® (paroxetine)
Prozac ® (fluoxetine)
Symbyax ®
Zoloft ® (sertraline)
SNRIs
Cymbalta ® (duloxetine)
Effexor ® (venlafaxine)
Pristiq ® (desvenlafaxine)
Tricyclics
Adapin ® (doxepin)
Anafranil ® (clomipramine)
Elavil ® (amitriptyline)
Endep ® (amitriptyline)
Ludiomil ® (maprotiline)
Norpramin ® (desipramine)
Pamelor ® (nortryptyline)
Pertofrane ® (desipramine)
Sinequan ® (doxepin)
Surmontil ® (trimipramine)
Tofranil ® (imipramine)
Vivactil ® (protriptyline)
Other Approved Anti-depressants
Remeron ® (mirtazapine)
Wellbutrin ® (bupropion)
Pending FDA Approval
Vilazodone ®

In other detailed embodiments of combinatorial formulations and coordinate treatment methods of the invention, examples of useful anti-depressant agents include, but are not limited to, one or more of the following: MAOIs, such as phenelzine, nortriptyline, selegiline and tranylcypromine; SSRIs, such as paroxetine, fluoxetine, citalopram, trazodone, fluvozamine and sertraline; Tricyclic anti-depressants, such as amitriptyline, desipramine, clomipramine, doxepine, trimipramine, amoxapine, protripyline and imipramine; Tetracyclic anti-depressants; Norepinephrine uptake inhibitors; Selective noradrenaline reuptake inhibitors; Serotonin and norepinephrine reuptake inhibitors, such as venlafaxine and duloxetine; and other anti-depressant agents such as maprotiline, nefazodone, and bupropion. In additional detailed embodiments the combinatorial formulations and coordinate treatment methods of the invention employ one or more useful psychotherapeutic agents selected from the following: SSRI's, such as Lexapro® (escitalopram HBr; indicated to treat depression and generalized anxiety disorder Celexa® (citalopram), Prozac®, Paxil®, Luvox® (fluvoxamine; also indicated to treat obsessive symptoms), Zoloft® (sertraline; also indicated to treat post-traumatic stress syndrome); Tricyclics, such as Amitriptyline, Desipramine, Nortriptyline; SSNRIs, such as Cymbalta® (Duloxetine), Effexor®, and desvenlafaxine; Tetracyclics, such as Remeron® (mirtazepine); MAOIs, such as Nardil® (phenelzine), and Parnate® (tranylcypromine); Serzone® (nefazodone; a phenylpiperazine); Trazodone® (a triazolopyridine); and Wellbutrin® (bupropion; an aminoketone). In additional detailed embodiments the combinatorial formulations and coordinate treatment methods of the invention employ one or more useful psychotherapeutic agents selected from the following: Amitriptyline; Amoxapine; Aripiprazole; Atomoxetine; Bupropion; Citalopram; Clomipramine; Desipramine; Desvenlafaxine; Dothiepin; Doxepin; Duloxetine; Escitalopram; Fluoxetine; Fluvoxamine; Imipramine; Isocarboxazid; Lofepramine; Maprotiline; Milnacipran; Mirtazapine; Moclobemide; Nefazodone; Notriptyline; Paroxetine; Phenelzine; Protriptyline; Quetiapine; Reboxetine; Selegiline; Sertraline; Tianeptine; Tranylcypromide; Trazodone; Trimipramine; and Venlafaxine.

In other detailed combinatorial formulations and coordinate treatment methods of the present invention, the psychotherapeutic agent is an anxiolytic drug agent including, but not limited to, benzodiazepines, such as alaprazolam, chlordiazepoxide, clonazepam, chlorazepate, diazepam, lorazepam, oxazepam and prazepam; non-benzodiazepine agents, such as buspirone; and tranquilizers, such as barbituates.

Benzodiazepines, anti-depressants, selective serotonin reuptake inhibitors and the azapirone agonist of the serotonin 1A receptor, buspirone (Lydiard et al., 1996) have been used with some success in the treatment of anxiety and anxiety disorders and are therefore contemplated for effective use within the methods and compositions of the invention.

The amount, timing and mode of delivery of compositions of the invention comprising an effective amount of a psychotherapeutic compound and an effective amount of a α and/or β blocker will be routinely adjusted on an individual basis, depending on such factors as weight, age, gender, and condition of the individual, the acuteness of the targeted anxiety disorder and/or related symptoms, whether the administration is prophylactic or therapeutic, and on the basis of other factors known to effect drug delivery, absorption, pharmacokinetics, including half-life, and efficacy.

In some embodiments, the compositions and formulations of the present invention may be administered according to a flexible dosing regimen. The treatment regimen provides for dosing periods during which a sufficient number of doses of the compositions and formulations of the present invention are administered to provide relief from anxiety symptoms. The one or more embodiments herein provides for dosing according to a discontinuous schedule. In a discontinuous schedule, each dosing period is followed by an evaluation period, during which the user can self-evaluate the occurrence and severity of symptoms. If the user determines that it is necessary to begin a new dosing period, the user may do so at any time following this evaluation period. If, however, the user feels the need to begin a new dosing period before the evaluation period has passed, or to take more than the recommended number of doses, then the user may, for example, choose to seek professional medical advice.

An effective dose or multi-dose treatment regimen for the psychotherapeutic compounds of the invention will ordinarily be selected to approximate a minimal dosing regimen that is necessary and sufficient to substantially prevent or alleviate one or more symptom(s) of the targeted anxiety disorder as described herein. For example, carvedilol may be administered in dosages ranging from 0.25 to 150 mg one or more times per day, preferably from 1 to 100 mg per day, and preferably from 2 to 50 mg per day. In some embodiments the dose of carvedilol may range from 0.5 to 30 mg per day, preferably 1 to 20 mg per day, preferably 3 to 16 mg per day, preferably 3.125 to 16.625 mg per day, and preferably 6.25 to 15.625 mg per day. Typically, carvedilol will be administered bi-daily. In some embodiments, sub-therapeutic amounts may be used. Exemplary suggested dosage ranges for selected drugs for use within certain embodiments of the invention are provided below, for illustrative purposes. Additional exemplary dosage ranges are provided below for selected drugs formulated for sustained delivery within additional embodiments of the invention, also for illustrative purposes.

### Exemplary Effective Dosage Ranges for Selected Oral Anti-Depressant Medications

| SSRIs | | |
|---|---|---|
| Celexa ® (citalopram) | 20-40 | mg |
| Lexapro ® (excitalopram oxalate) | 10-20 | mg |
| Luvox ® (fluvoxamine) | 100 | mg |
| Paxil ® (paroxetine) | 20-40 | mg |
| Prozac ® (fluoxetine) | 20 | mg |
| Symbyax ® (Zyprexa & Prozac) | 12.5-50 | mg |
| Zoloft ® (sertraline) | 50-200 | mg |
| SNRIs | | |
| Cymbalta ® (duloxetine) | 60-120 | mg |
| Effexor ® (venlafaxine) | 75-375 | mg |
| Pristiq ® (desvenlafaxine) | 50 | mg |
| Tricyclics | | |
| Adapin ® (doxepin) | 150 | mg |
| Anafranil ® (clomipramine) | 150 | mg |
| Elavil ® (amitriptyline) | 150 | mg |
| Endep ® (amitriptyline) | 150 | mg |
| Ludiomil ® (maprotiline) | 100 | mg |
| Norpramin ® (desipramine) | 150 | mg |
| Pamelor ® (nortryptyline) | 75 | mg |
| Pertofrane ® (desipramine) | 150 | mg |
| Sinequan ® (doxepin) | 150 | mg |
| Surmontil ® (trimipramine) | 150 | mg |
| Tofranil ® (imipramine) | 150 | mg |
| Vivactil ® (protriptyline) | 75 | mg |
| Other Approved Anti-depressants | | |
| Remeron ® (mirtazapine) | 20-40 | mg |
| Wellbutrin ® (bupropion) | 300 | mg |
| Pending FDA Approval | | |
| Vilazodone ® | 40 | mg |

### Exemplary Effective Dosage Ranges for Selected Psychotherapeutic Compounds in Sustained Release Formulations/Methods

| SSRIs | | |
|---|---|---|
| Celexa ® (citalopram) | 60-1000 | mg |
| Lexapro ® (excitalopram oxalate) | 60-1000 | mg |
| Luvox ® (fluvoxamine) | 100-2000 | mg |
| Paxil ® (paroxetine) | 60-100 | mg |
| Prozac ® (fluoxetine) | 60-50 | mg |
| Symbyax ® (Zyperxa & Prozac) | 60-500 | mg zyprexa |
| | 60-1000 | mg prozac |
| Zoloft ® (sertraline) | 200-2500 | mg |
| SNRIs | | |
| Cymbalta ® (duloxetine) | 100-2000 | mg |
| Effexor ® (venlafaxine) | 300-3000 | mg |
| Pristiq ® (desvenlafaxine) | 100-2000 | mg |
| Tricyclics | | |
| Adapin ® (doxepin) | 200-1000 | mg |
| Anafranil ® (clomipramine) | 100-1000 | mg |
| Elavil ® (amitriptyline) | 150-2000 | mg |
| Endep ® (amitriptyline) | 150-2000 | mg |
| Ludiomil ® (maprotiline) | 100-1000 | mg |
| Norpramin ® (desipramine) | 100-1000 | mg |
| Pamelor ® (nortryptyline) | 100-1000 | mg |
| Pertofrane ® (desipramine) | 100-1000 | mg |
| Sinequan ® (doxepin) | 100-1000 | mg |
| Surmontil ® (trimipramine) | 100-1000 | mg |
| Tofranil ® (imipramine) | 100-100 | mg |
| Vivactil ® (protriptyline) | 50-500 | mg |
| Other Approved Anti-depressants | | |
| Remeron ® (mirtazapine) | 50-500 | mg |
| Wellbutrin ® (bupropion) | 300-3000 | mg |
| Pending FDA Approval | | |
| Vilazodone ® | 50-1000 | mg |

These and other effective unit dosage amounts of either or both of the psychotherapeutic agent and/or α and/or β blocker may be administered in a single dose, or in the form of multiple daily, weekly or monthly doses, for example in a dosing regimen comprising from 1 to 5, or 2-3, doses administered per day, per week, or per month. In exemplary embodiments, exemplary dosages of selected drugs as illustrated above are administered one, two, three, or four times per day. In more detailed embodiments, specific dosages within the specified exemplary ranges above are administered once, twice, or three times daily. In alternate embodiments, dosages are calculated based on body weight, and may be administered, for example, in amounts as exemplified above adjusted for body weight.

Pharmaceutical dosage forms of a compound of the present invention may optionally include excipients recognized in the art of pharmaceutical compounding as being suitable for the preparation of dosage units as discussed above. Such excipients include, without intended limitation, binders, fillers, lubricants, emulsifiers, suspending agents, sweeteners, flavorings, preservatives, buffers, wetting agents, disintegrants, effervescent agents and other conventional excipients and additives.

The compositions of the invention for treating anxiety disorders, including PTSD, can thus include any one or combination of the following: a pharmaceutically acceptable carrier or excipient; other medicinal agent(s); pharmaceutical agent(s); adjuvants; buffers; preservatives; diluents; and various other pharmaceutical additives and agents known to those skilled in the art. These additional formulation additives and agents will often be biologically inactive and can be administered to patients without causing deleterious side effects or interactions with the active agent.

As used herein, an “active therapeutic agent” of the present invention includes a combined α/β blocker compound, or α and/or β blocker, and optionally includes a psychotherapeutic compound.

An active therapeutic agent of the present invention will often be formulated and administered in an oral dosage form, optionally in combination with a carrier or other additive(s). Suitable carriers common to pharmaceutical formulation technology include, but are not limited to, microcrystalline cellulose, lactose, sucrose, fructose, glucose dextrose, or other sugars, di-basic calcium phosphate, calcium sulfate, cellulose, methylcellulose, cellulose derivatives, kaolin, mannitol, lactitol, maltitol, xylitol, sorbitol, or other sugar alcohols, dry starch, dextrin, maltodextrin or other polysaccharides, inositol, or mixtures thereof. Exemplary unit oral dosage forms for use in this invention include tablets and capsules, which may be prepared by any conventional method of preparing pharmaceutical oral unit dosage forms can be utilized in preparing oral unit dosage forms. Oral unit dosage forms, such as tablets or capsules, may contain one or more conventional additional formulation ingredients, including, but are not limited to, release modifying agents, glidants, compression aides, disintegrants, lubricants, binders, flavors, flavor enhancers, sweeteners and/or preservatives. Suitable lubricants include stearic acid, magnesium stearate, talc, calcium stearate, hydrogenated vegetable oils, sodium benzoate, leucine carbowax, magnesium lauryl sulfate, colloidal silicon dioxide and glyceryl monostearate. Suitable glidants include colloidal silica, fumed silicon dioxide, silica, talc, fumed silica, gypsum and glyceryl monostearate. Substances which may be used for coating include hydroxypropyl cellulose, titanium oxide, talc, sweeteners and colorants. The aforementioned effervescent agents and disintegrants are useful in the formulation of rapidly disintegrating tablets known to those skilled in the art. These typically disintegrate in the mouth in less than one minute, and preferably in less than thirty seconds. By effervescent agent is meant a couple, typically an organic acid and a carbonate or bicarbonate. Such rapidly acting dosage forms would be useful, for example, in the prevention or treatment of acute attacks of panic disorder.

The active therapeutic agent of the invention can be prepared and administered in any of a variety of inhalation or nasal delivery forms known in the art. Devices capable of depositing aerosolized formulations of an active therapeutic agent of the invention in the sinus cavity or pulmonary alveoli of a patient include metered dose inhalers, nebulizers, dry powder generators, sprayers, and the like. Pulmonary delivery to the lungs for rapid transit across the alveolar epithelium into the blood stream may be particularly useful in treating impending episodes of seizures or panic disorder. Methods and compositions suitable for pulmonary delivery of drugs for systemic effect are well known in the art. Suitable formulations, wherein the carrier is a liquid, for administration, as for example, a nasal spray or as nasal drops, may include aqueous or oily solutions of a compound of the present invention, and any additional active or inactive ingredient(s).

Intranasal delivery permits the passage of active compounds of the invention into the blood stream directly after administering an effective amount of the compound to the nose, without requiring the product to be deposited in the lung. In addition, intranasal delivery can achieve direct, or enhanced, delivery of the active therapeutic agent to the central nervous system (CNS). In these and other embodiments, intranasal administration of the compounds of the invention may be advantageous for treating a variety of CNS disorders, including anxiety, by providing for rapid absorption and CNS delivery.

For intranasal and pulmonary administration, a liquid aerosol formulation will often contain an active compound of the invention combined with a dispersing agent and/or a physiologically acceptable diluent. Alternative, dry powder aerosol formulations may contain a finely divided solid form of the subject compound and a dispersing agent allowing for the ready dispersal of the dry powder particles. With either liquid or dry powder aerosol formulations, the formulation must be aerosolized into small, liquid or solid particles in order to ensure that the aerosolized dose reaches the mucous membranes of the nasal passages or the lung. The term “aerosol particle” is used herein to describe a liquid or solid particle suitable of a sufficiently small particle diameter, e.g., in a range of from about 2-5 microns, for nasal or pulmonary distribution to targeted mucous or alveolar membranes. Other considerations include the construction of the delivery device, additional components in the formulation, and particle characteristics. These aspects of nasal or pulmonary administration of drugs are well known in the art, and manipulation of formulations, aerosolization means, and construction of delivery devices, is within the level of ordinary skill in the art.

Yet additional compositions and methods of the invention are provided for topical administration of an active therapeutic agent of the present invention for treating anxiety disorders including PTSD. Topical compositions may comprise a compound of the present invention and any other active or inactive component(s) incorporated in a dermatological or mucosal acceptable carrier, including in the form of aerosol sprays, powders, dermal patches, sticks, granules, creams, pastes, gels, lotions, syrups, ointments, impregnated sponges, cotton applicators, or as a solution or suspension in an aqueous liquid, non-aqueous liquid, oil-in-water emulsion, or water-in-oil liquid emulsion. These topical compositions may comprise a compound of the present invention dissolved or dispersed in water or other solvent or liquid to be incorporated in the topical composition or delivery device. It can be readily appreciated that the transdermal route of administration may be enhanced by the use of various dermal penetration enhancers known to those skilled in the art. Formulations suitable for such dosage forms incorporate excipients commonly utilized therein, particularly means, e.g. structure or matrix, for sustaining the absorption of the drug over an extended period of time, for example 24 hours. A once-daily transdermal patch will be particularly useful for patients suffering from or at risk for selected anxiety disorders, such as generalized anxiety disorder or PTSD.

Use of transdermal delivery devices and methods is particularly advantageous for administration of carvedilol within the present invention. While orally administered carvedilol is rapidly absorbed from the gastrointestinal tract, its oral bioavailability remains low, at approximately 23%, due to significant first-pass hepatic metabolism (Ubaidulla et al., 2007). Transdermal administration of carvedilol may provide several advantages over oral delivery, including improved patient compliance and responsivity, bypass of first-pass metabolism, sustained drug delivery, and minimal variability both within and between patients. The properties of carvedilol indicate that it is suitable for formulation in a transdermal patch. These properties include a low molecular weight (406.5), a favorable logarithmic partition coefficient (log octanol/water: 0.58±0.02; log octanol/buffer pH 7.4: 0.61±0.06), smaller dose range (1-50 mg), short plasma half-life (approximately 6 hours), and poor oral bioavailability (Ubaidulla et al., 2007).

A matrix-type transdermal patch developed for carvedilol treatment of hypertension was found to be effective in a study in a rat model of hypertension (Ubaidulla et al., 2007). In that study, carvedilol was found to be compatible with polymers commonly used in transdermal patches. Moreover, the carvedilol patches delivered consistent plasma concentrations of carvedilol with improved bioavailability of 62-71% in comparison to orally administered carvedilol, which has reported bioavailability of 23% (Ubaidulla et al., 2007). The results of that study indicate that transdermal patches of carvedilol will be effective within the compositions and methods of the present invention for treatment of anxiety disorders and PTSD.

Accordingly, a matrix-type transdermal patch is a preferred embodiment for carvedilol treatment of anxiety disorders and PTSD. The dose of carvedilol delivered by transdermal patch will typically range from 0.5-50 mg per day, more often from 1.0 to 30 mg per day, 1 to 20 mg per day, or 3 to 15 mg per day, as determined by the prescribing physician in light of such ordinary dosing factors as patient condition, body weight, duration of treatment, etc. Transdermal patch delivery devices and methods of the invention will yield increased carvedilol bioavailability compared to oral administration of an equivalent carvedilol dose. In particular, a transdermal patch will result in a 20-50% bioavailability increase, more typically a 50%-100% increase, up to a two-fold, three-fold, or greater increase, and as much as a five-fold or greater increase in bioavailability compared to that obtained with an equivalent oral dosage form (e.g., an IR or SR capsule or tablet form). The pharmacokinetic properties of oral and transdermal patch delivered carvedilol can be measured as described in Example 6. Within more detailed aspects of the invention, transdermal delivery devices and methods for delivering carvedilol and other therapeutic compounds as described herein will yield approximately zero-order kinetics, to provide steady state levels of the active drug within 24 hours, and to reduce peak to trough ratios of drug levels (e.g., reduced Cₘₐₓ/Cₘᵢₙ) in comparison to oral delivery, to yield more continuous therapeutic exposure. In exemplary embodiments, the Cₘₐₓ will be roughly equivalent for the two forms of administration, however the Cₘₐₓ from comparative oral administration will be reached at about 2 hours, while that of the transdermal administration will be reached in a more extended, attenuated time frame, e.g., in greater than four hours, from 4-6 hours, more typically from 6-18 hours, and in exemplary embodiments in about 10-12 hours, thereby yielding substantial therapeutic advantages over oral delivery. The transdermal administration of carvedilol will provide the above-described increase in bioavailability compared to oral administration, e.g., as determined by plasma concentration measurements and expressed as an increase in comparative AUCs obtained with transdermal versus administration at pre-determined time intervals. Since low bioavailability results in increased variability in drug exposure among patients, the use of the transdermal delivery devices and methods of the invention yield more consistent drug levels, and associated normalized and improved pharmacodynamic actions and therapeutic benefits among treated patients.

Transdermal delivery systems useful for the compositions and methods of the present invention are typically fabricated as multilayered polymeric laminates in which a drug reservoir or a drug-polymer matrix is sandwiched between two polymeric layers: an outer impervious backing layer that creates an occlusive environment and prevents the loss of drug through the backing surface and an inner polymeric layer that functions as an adhesive and/or rate-controlling membrane. In the case of a drug reservoir design, the reservoir is sandwiched between the backing and a rate controlling membrane. The drug releases only through the rate-controlling membrane, which can be microporous or nonporous. In the drug reservoir compartment, the drug can be in the form of a solution, suspension, or gel or dispersed in a solid polymer matrix. On the outer surface of the polymeric membrane a thin layer of drug-compatible, hypoallergenic adhesive polymer may be applied.

For the drug matrix design, two general types of system include the drug-in-adhesive system and the matrix dispersion system. In the drug-in-adhesive system, the drug reservoir is formed by dispersing the drug in an adhesive polymer and then spreading the medicated polymer adhesive by solvent casting or by melting the adhesive (in the case of hot-melt adhesives) onto an impervious backing layer. On top of the reservoir, layers of unmedicated adhesive polymer are applied. In the case of the matrix dispersion system, the drug is dispersed homogeneously in a hydrophilic or lipophilic polymer matrix and fixed onto a drug-impermeable backing layer by solvent casting or extrusion. Instead of applying the adhesive on the face of the drug reservoir, it is applied to form a peripheral adhesive. Illustrative examples of suitable adhesives as matrix type delivery systems include those described in U.S. Pat. Nos. 5,474,783, and 5,656,386. Other transdermal systems include films, plasters, dressings, and bandages, as well as multilayer delivery systems in which the drug is solubilized or contained in one or more separate layers, and reservoir-type delivery systems in which the drug is solubilized or contained in a reservoir or depot separate from the adhesive which attaches directly to the skin or mucosa.

Suitable adhesives for transdermal patches are known in the art and include pressure-sensitive adhesives and bioadhesives. Pressure sensitive adhesives suitable for use in accordance with the invention include, but are not limited to, pressure-sensitive silicone adhesives, pressure-sensitive acrylic adhesives, and mixtures of any two or more thereof. Exemplary pressure-sensitive silicone adhesives include polysiloxanes and other silicone adhesives as disclosed in U.S. Pat. Nos. 4,591,622; 4,584,355; 4,585,836; 4,655,767; and 5,958,446. Suitable silicone pressure-sensitive adhesives are commercially available and include the silicone adhesives sold under the trademarks BIO-PSA X7-3027. BIO-PSA X7-4919, BIO-PSA X7-2685, and BIO-PSA X7-3122 by Dow Corning Corporation, Medical Products, Midland, Mich.

Bioadhesive materials useful in some embodiments include those described in U.S. Pat. No. 6,562,363. For example, bioadhesive materials may include polymers, either water soluble or water insoluble, with or without crosslinking agents, which are bioadhesive. Exemplary bioadhesives include natural materials, cellulose materials, synthetic and semi-synthetic polymers, and generally, any physiologically acceptable polymer showing bioadhesive properties, or mixtures of any two or more thereof.

Suitable acrylic-based pressure-sensitive adhesives for transdermal patches are also known in the art. Such acrylic-based polymers may be used as the primary pressure-sensitive adhesive (see, e.g., U.S. Pat. No. 4,390,520), or may be used in combination with other polymers which may or may not be pressure-sensitive adhesives (see, e.g. U.S. Pat. No. 4,994,267). Acrylic-based pressure-sensitive adhesives may be polymerized with functional monomers to provide functional groups on the acrylic-based adhesive, such as may be desired to improve wear properties and drug delivery. Suitable polyacrylic acid polymers include polymers of acrylic acid crosslinked with polyalkenenyl ethers (generically known as carbomers) or divinyl glycol (generically known as polycarbophils).

Polymer blends as described in U.S. Pat. No. 5,958,446 may also be used as pharmaceutically acceptable carriers and adhesives in the transdermal compositions embodied herein.

In certain embodiments of transdermal patches used in the compositions of the invention, a plasticizer or tackifying agent is incorporated into the formulation to improve the adhesive characteristics of the composition. A tackifying agent is particularly useful in those embodiments in which the drug does not plasticize the polymer. Suitable tackifying agents are those known in the art including: (1) aliphatic hydrocarbons; (2) mixed aliphatic and aromatic hydrocarbons; (3) aromatic hydrocarbons; (4) substituted aromatic hydrocarbons; (5) hydrogenated esters; (6) polyterpenes; and (7) hydrogenated wood rosins. The tackifying agent employed is preferably compatible with the blend of polymers. In some embodiments, the tackifying agent is silicone fluid (e.g., 360 Medical Fluid, available from Dow Corning Corporation, Midland, Mich.) or mineral oil. Silicone fluid is useful for blends comprising polysiloxane as a major component. In other embodiments, where polyacrylate, for example, is a major component, mineral oil may be used as a tackifying agent.

Transdermal patch compositions may also contain agents known to accelerate the delivery of the drug through the skin. Such agents have been referred to as skin-penetration enhancers, accelerants, adjuvants, and sorption promoters. This class of agents includes those with diverse mechanisms of action including those which have the function of improving the solubility and diffusibility of the drug within the multiple polymer and those which improve percutaneous absorption, for example, by changing the ability of the stratum corneum to retain moisture, softening the skin, improving the skin's permeability, acting as penetration assistants or hair-follicle openers or changing the state of the skin including the boundary layer. Some of these agents have more than one mechanism of action, but in essence they serve to enhance the delivery of the drug. Some exemplary agents are listed in U.S. Pat. Nos. 5,958,446 and 6,562,363.

The topical or transdermal compositions of the present invention can be made in accordance with methods known in the art. For example, carvedilol can be blended with the pharmaceutically acceptable topical or transdermal carrier components, or by dissolving the carvedilol in a solvent and combining the solution with the pharmaceutically acceptable topical or transdermal carrier components, or by other conventional methods. In the case of a transdermal patch, the substrate is laminated to one or more additional layers, such as a protective layer, a backing layer, a rate-controlling layer, a membrane layer, or one or more other types of layers known in the art.

Yet additional formulations of a compound of the present invention are provided for parenteral administration, including aqueous and non-aqueous sterile injection solutions which may optionally contain anti-oxidants, buffers, bacteriostats and/or solutes which render the formulation isotonic with the blood of the mammalian subject; aqueous and non-aqueous sterile suspensions which may include suspending agents and/or thickening agents; dispersions; and emulsions. The formulations may be presented in unit-dose or multi-dose containers. Pharmaceutically acceptable formulations and ingredients will typically be sterile or readily sterilizable, biologically inert, and easily administered. Parenteral preparations typically contain buffering agents and preservatives, and may be lyophilized for reconstitution at the time of administration.

Parental formulations may also include polymers for extended release following parenteral administration. Such polymeric materials are well known to those of ordinary skill in the pharmaceutical compounding arts. Extemporaneous injection solutions, emulsions and suspensions may be prepared from sterile powders, granules and tablets of the kind previously described. Preferred unit dosage formulations are those containing a daily dose or unit, daily sub-dose, as described herein above, or an appropriate fraction thereof, of the active ingredient(s).

In further embodiments, formulations of a compound of the present invention are provided for intramuscular administration.

Within exemplary compositions and dosage forms of the invention, the active therapeutic agent for treating anxiety disorders is/are administered in an extended release or sustained release formulation. In these formulations, the sustained release composition of the formulation provides therapeutically effective plasma levels of the active therapeutic agent over a sustained delivery period of approximately 8 hours or longer, or over a sustained delivery period of approximately 18 hours or longer, up to a sustained delivery period of approximately 24 hours or longer.

In exemplary embodiments, the active therapeutic agent is/are combined with a sustained release vehicle, matrix, binder, or coating material. As used herein, the term “sustained release vehicle, matrix, binder, or coating material” refers to any vehicle, matrix, binder, or coating material that effectively, significantly delays dissolution of the active compound in vitro, and/or delays, modifies, or extends delivery of the active compound into the blood stream (or other in vivo target site of activity) of a subject following administration (e.g., oral administration), in comparison to dissolution and/or delivery provided by an “immediate release” formulation, as described herein, of the same dosage amount of the active compound. Accordingly, the term “sustained release vehicle, matrix, binder, or coating material” as used herein is intended to include all such vehicles, matrices, binders and coating materials known in the art as “sustained release”, “delayed release”, “slow release”, “extended release”, “controlled release”, “modified release”, and “pulsatile release” vehicles, matrices, binders and coatings.

In one aspect, the current invention comprises an oral sustained release dosage composition for administering an active therapeutic agent according to the invention. In a related aspect, the invention comprises a method of reducing one or more side effects that attend administration of an oral dosage form of an active therapeutic agent by employing a sustained release formulation. Within these methods, an active therapeutic agent is provided in a sustained release oral dosage form and the dosage form is introduced into a gastrointestinal tract of a mammalian subject presenting with an anxiety disorder amenable to treatment using the subject, by having the subject swallow the dosage form. The method further includes releasing the active therapeutic agent in a sustained, delayed, gradual or modified release delivery mode into the gastrointestinal tract (e.g., the intestinal lumen) of the subject over a period of hours, during which the active therapeutic agent reach(es), and is/are sustained at, therapeutic concentration(s) in a blood plasma, tissue, organ or other target site of activity (e.g., a central nervous system (CNS) tissue, fluid or compartment) in the patient. When following this method, the side effect profile of the active therapeutic agent is less than a side effect profile of an equivalent dose of the active therapeutic agent administered in an immediate release oral dosage form.

In certain embodiments, the active therapeutic agent is/are released from the sustained release compositions and dosage forms of the invention and delivered into the blood plasma or other target site of activity in the subject at a sustained therapeutic level over a period of at least about 6 hours, often over a period of at least about 8 hours, at least about 12 hours, or at least about 18 hours, and in other embodiments over a period of about 24 hours or greater. By sustained therapeutic level is meant a plasma concentration level of at least a lower end of a therapeutic dosage range as exemplified herein. In more detailed embodiments of the invention, the sustained release compositions and dosage forms will yield a therapeutic level of an active therapeutic agent following administration to a mammalian subject in a desired dosage amount (e.g., 25, 50, 100, or 200 mg) that yields a minimum plasma concentration of at least a lower end of a therapeutic dosage range as exemplified herein over a period of at least about 6 hours, at least about 8 hours, at least about 12 hours, at least about 18 hours, or up to 24 hours or longer. In alternate embodiments of the invention, the sustained release compositions and dosage forms will yield a therapeutic level of active therapeutic agent following administration to a mammalian subject in a desired dosage amount (e.g., 25, 50, 100, or 200 mg) that yields a minimum plasma concentration that is known to be associated with clinical efficacy, over a period of at least about 6 hours, at least about 8 hours, at least about 12 hours, at least about 18 hours, or up to 24 hours or longer.

In certain embodiments, the active therapeutic agent is/are released from the compositions and dosage forms of the invention and delivered into the blood plasma or other target site of activity in the subject in a sustained release profile characterized in that from about 0% to 20% of the active compound is released and delivered (as determined, e.g., by measuring blood plasma levels) within in 0 to 2 hours, from 20% to 50% of the active compound is released and delivered within about 2 to 12 hours, from 50% to 85% of the active compound is released and delivered within about 3 to 20 hours, and greater than 75% of the active compound is released and delivered within about 5 to 18 hours.

In more detailed embodiments of the invention, compositions and oral dosage forms of an active therapeutic agent are provided, wherein the compositions and dosage forms, after ingestion, provide a curve of concentration of the active therapeutic agent over time, the curve having an area under the curve (AUC) which is approximately proportional to the dose of the active therapeutic agent administered, and a maximum concentration (Cmax) that is proportional to the dose of the active therapeutic agent administered.

In other detailed embodiments, the Cmax of the active therapeutic agent provided after oral delivery of a composition or dosage form of the invention is less than about 80%, often less than about 75%, in some embodiments less than about 60%, or 50%, of a Cmax obtained after administering an equivalent dose of the active compound in an immediate release oral dosage form.

In other detailed embodiments, the AUC of the active therapeutic agent provided after oral delivery of a composition or dosage form of the invention is less than about 80%, often less than about 75%, in some embodiments less than about 60%, or 50%, of a AUC obtained after administering an equivalent dose of the active compound in an immediate release oral dosage form.

In other detailed embodiments, each of the Cmax and AUC of the active therapeutic agent provided after oral delivery of a composition or dosage form of the invention is less than about 80%, often less than about 75%, in some embodiments less than about 60%, or 50%, of a Cmax and AUC obtained after administering an equivalent dose of the active compound in an immediate release oral dosage form.

Within exemplary embodiments of the invention, the compositions and dosage forms containing the active therapeutic agent and a sustained release vehicle, matrix, binder, or coating will yield sustained delivery of the active compound such that, following administration of the composition or dosage form to a mammalian treatment subject, the Cmax of the active therapeutic agent in the treatment subject is less than about 80% of a Cmax provided in a control subject after administration of the same amount of the active therapeutic agent in an immediate release formulation.

Within other exemplary embodiments of the invention, the compositions and dosage forms containing the active therapeutic agent and a sustained release vehicle, matrix, binder, or coating will yield sustained delivery of the active compound such that, following administration of the composition or dosage form to a mammalian treatment subject, the AUC of the active compound in the treatment subject is less than about 80% of a AUC provided in a control subject after administration of the same amount of the active agent in an immediate release formulation.

Within additional exemplary embodiments, the compositions and dosage forms containing the active therapeutic agent and a sustained release vehicle, matrix, binder, or coating will yield sustained delivery of the active compound such that, following administration of the composition or dosage form to a mammalian treatment subject, the Cmax and AUC of the active compound in the treatment subject are, respectively, less than about 80% of a Cmax and a AUC provided in a control subject after administration of the same amount of the active agent in an immediate release formulation

As used herein, the term “immediate release dosage form” refers to a dosage form of an active therapeutic agent wherein the active compound readily dissolves upon contact with a liquid physiological medium, for example phosphate buffered saline (PBS) or natural or artificial gastric fluid. In certain embodiments, an IR formulation will be characterized in that at least 70% of the active compound will be dissolved within a half hour after the dosage form is contacted with a liquid physiological medium. For example, at least 70% of the active compound in an IR α and/or β blocker dosage form will dissolve within a half hour following contact of the dosage form with a liquid physiological medium in an art-accepted in vitro dissolution assay (e.g., using a USP 1 Apparatus, 20 mesh baskets, 75 rpm, and a dissolution medium comprised of 900 ml 0.01 N HCl at 37° C.±0.5° C.; or following an alternate USP basket method at 100 rpm in 700 ml Simulated Gastric Fluid (SGF) at 37° C. for 1 hour and thereafter switching to 900 ml with phosphate buffer to a pH of 7.5 at 37° C.). In alternate embodiments, at least 80%, 85%, 90% or more, or up to 100%, of the active compound in an IR dosage form will dissolve within a half hour following contact of the dosage form with a liquid physiological medium in an art-accepted in vitro dissolution assay. These general characteristics of an IR dosage form will often relate to powdered or granulated compositions of an active therapeutic agent in a capsulated dosage form, for example in a gelatin-encapsulated dosage form, where dissolution will often be relatively immediate after dissolution/failure of the gelatin capsule. In alternate embodiments, the IR dosage form may be provided in the form of a compressed tablet, granular preparation, powder, or even liquid dosage form, in which cases the dissolution profile will often be even more immediate (e.g., wherein at least 85%-95% of the active compound is dissolved within a half hour).

In additional embodiments of the invention, an IR dosage form will include compositions wherein the active therapeutic agent is not admixed, bound, coated or otherwise associated with a formulation component that substantially impedes in vitro or in vivo dissolution and/or in vivo bioavailability of the active compound. Within certain embodiments, the active therapeutic agent will be provided in an immediate release dosage form that does not contain significant amounts of a sustained release vehicle, matrix, binder or coating material. In this context, the term “significant amounts of a sustained release vehicle, matrix, binder or coating material” is not intended to exclude any amount of such materials, but an amount sufficient to impede in vitro or in vivo dissolution of an active therapeutic agent in a formulation containing such materials by at least 5%, often at least 10%, and up to at least 15%-20% compared to dissolution of the active therapeutic agent when provided in a composition that is essentially free of such materials.

In alternate embodiments of the invention, an IR dosage form of a psychotherapeutic therapeutic compound and/or α and/or β blocker may be any dosage form comprising the active compound which fits the FDA Biopharmaceutics Classification System (BCS) Guidance definition (see, e.g., http://www.fda.gov/cder/OPS/BCS_guidance.htm) of a “high solubility substance in a rapidly dissolving formulation”. In exemplary embodiments, an IR formulation of an active therapeutic agent formulation according to this aspect of the invention will exhibit rapid dissolution characteristics according to BCS Guidance parameters, such that at least approximately 85% of the active therapeutic agent in the formulation will go into a test solution within about 30 minutes at pH 1, pH 4.5, and pH 6.8.

The compositions, dosage forms and methods of the invention thus include novel tools for coordinate treatment of anxiety disorders providing for sustained release and/or sustained delivery of the active therapeutic agent. As used herein, “sustained release” and “sustained delivery” are evinced by a sustained, delayed, extended, or modified, in vitro or in vivo dissolution rate, in vivo release and/or delivery rate, and/or in vivo pharmacokinetic value(s) or profile.

Within exemplary embodiments of the invention, the sustained release and sustained delivery compositions and dosage forms of the invention will exhibit less than about 80% of one or more release/delivery property(ies) value(s) or range(s) selected from i) an in vitro dissolution rate, ii) in vivo dissolution or release rate, and/or iii) plasma Cmax, AUC, and/or Cmax and AUC, exhibited by an otherwise comparable, immediate release composition or dosage form of the active compound. Often, the one or more release/delivery property(ies) selected from i) an in vitro dissolution rate, ii) in vivo dissolution or release rate, and/or iii) plasma Cmax, AUC, and/or Cmax and AUC of the sustained release compositions and dosage forms of the invention will be less than about 75%, in some embodiments less than about 60%, or 50%, of the respective release/delivery property(ies) of an otherwise comparable, immediate release dosage form of the active compound. The terms “sustained release” and “sustained delivery” are intended herein to encompass release and delivery properties conventionally known in the art as “sustained”, “delayed”, “slow”, “extended”, “controlled”, “modified”, and “pulsatile” release and delivery.

The sustained release dosage forms of the present invention can take any form as long as one or more of the dissolution, release, delivery and/or pharmacokinetic property(ies) identified above are satisfied. Within illustrative embodiments, the composition or dosage form can comprise an active therapeutic agent combined with any one or combination of: a drug-releasing polymer, matrix, bead, microcapsule, or other solid drug-releasing vehicle; drug-releasing tiny timed-release pills or mini-tablets; compressed solid drug delivery vehicle; controlled release binder; multi-layer tablet or other multi-layer or multi-component dosage form; drug-releasing lipid; drug-releasing wax; and a variety of other sustained drug release materials as contemplated herein, or formulated in an osmotic dosage form.

The present invention thus provides a broad range of sustained release compositions and dosage forms comprising an active therapeutic agent, which in certain embodiments are adapted for providing sustained release of the active compound(s) following, e.g., oral administration. Sustained release vehicles, matrices, binders and coatings for use in accordance with the invention include any biocompatible sustained release material which is inert to the active agent and which is capable of being physically combined, admixed, or incorporated with the active compound. Useful sustained release materials may be dissolved, degraded, disintegrated, and/or metabolized slowly under physiological conditions following delivery (e.g., into a gastrointestinal tract of a subject, or following contact with gastric fluids or other bodily fluids). Useful sustained release materials are typically non-toxic and inert when contacted with fluids and tissues of mammalian subjects, and do not trigger significant adverse side effects such as irritation, immune response, inflammation, or the like. They are typically metabolized into metabolic products which are biocompatible and easily eliminated from the body.

In certain embodiments, sustained release polymeric materials are employed as the sustained release vehicle, matrix, binder, or coating (see, e.g., “Medical Applications of Controlled Release,” Langer and Wise (eds.), CRC Press., Boca Raton, Fla. (1974); “Controlled Drug Bioavailability,” Drug Product Design and Performance, Smolen and Ball (eds.), Wiley, N.Y. (1984); Ranger and Peppas, 1983, J Macromol. Sci. Rev. Macromol Chem. 23:61; see also Levy et al., 1985, Science 228: 190; During et al., 1989, Ann. Neurol. 25:351; Howard et al, 1989, J. Neurosurg. 71:105, each incorporated herein by reference). Within exemplary embodiments, useful polymers for co-formulating with the active therapeutic agent to yield a sustained release composition or dosage form include, but are not limited to, ethylcellulose, hydroxyethyl cellulose; hydroxyethylmethyl cellulose; hydroxypropyl cellulose; hydroxypropylmethyl cellulose; hydroxypropylmethyl cellulose phthalate; hydroxypropylmethylcellulose acetate succinate; hydroxypropylmethylcellulose acetate phthalate; sodium carboxymethylcellulose; cellulose acetate phthalate; cellulose acetate trimellitate; polyoxyethylene stearates; polyvinyl pyrrolidone; polyvinyl alcohol; copolymers of polyvinyl pyrrolidone and polyvinyl alcohol; polymethacrylate copolymers; and mixtures thereof.

Additional polymeric materials for use as sustained release vehicles, matrices, binders, or coatings within the compositions and dosage forms of the invention include, but are not limited to, additional cellulose ethers, e.g., as described in Alderman, Int. J. Pharm. Tech. & Prod. Mfr., 1984, 5(3) 1-9 (incorporated herein by reference). Other useful polymeric materials and matrices are derived from co-polymeric and homopolymeric polyesters having hydrolysable ester linkages. A number of these are known in the art to be biodegradable and to lead to degradation products having no or low toxicity. Exemplary polymers in this context include polyglycolic acids (PGAs) and polylactic acids (PLAs), poly(DL-lactic acid-co-glycolic acid) (DL PLGA), poly(D-lactic acid-coglycolic acid) (D PLGA) and poly(L-lactic acid-co-glycolic acid) (L PLGA). Other biodegradable or bioerodable polymers for use within the invention include such polymers as poly(ε-caprolactone), poly(ε-aprolactone-CO-lactic acid), poly(ε-aprolactone-CO-glycolic acid), poly(β-hydroxy butyric acid), poly(alkyl-2-cyanoacrilate), hydrogels such as poly(hydroxyethyl methacrylate), polyamides, poly-amino acids (e.g., poly-L-leucine, poly-glutamic acid, poly-L-aspartic acid, and the like), poly(ester ureas), poly(2-hydroxyethyl DL-aspartamide), polyacetal polymers, polyorthoesters, polycarbonates, polymaleamides, polysaccharides, and copolymers thereof. Methods for preparing pharmaceutical formulations using these polymeric materials are generally known to those skilled in the art (see, e.g., Sustained and Controlled Release Drug Delivery Systems, J. R. Robinson, ed., Marcel Dekker, Inc., New York, 1978, incorporated herein by reference).

In other embodiments of the invention, the compositions and dosage forms comprise an active therapeutic agent coated on a polymer substrate. The polymer can be an erodible or a nonerodible polymer. The coated substrate may be folded onto itself to provide a bilayer polymer drug dosage form. For example the active therapeutic agent can be coated onto a polymer such as a polypeptide, collagen, gelatin, polyvinyl alcohol, polyorthoester, polyacetyl, or a polyorthocarbonate, and the coated polymer folded onto itself to provide a bilaminated dosage form. In operation, the bioerodible dosage form erodes at a controlled rate to dispense the active compound over a sustained release period. Representative biodegradable polymers for use in this and other aspects of the invention can be selected from, for example, biodegradable poly(amides), poly(amino acids), poly(esters), poly(lactic acid), poly(glycolic acid), poly(carbohydrate), poly(orthoester), poly(orthocarbonate), poly(acetyl), poly(anhydrides), biodegradable poly(dehydropyrans), and poly(dioxinones) which are known in the art (see, e.g., Rosoff, Controlled Release of Drugs, Chap. 2, pp. 53-95 (1989); and U.S. Pat. Nos. 3,811,444; 3,962,414; 4,066,747, 4,070,347; 4,079,038; and 4,093,709, each incorporated herein by reference).

In another embodiment of the invention, the dosage form comprises an active therapeutic agent loaded into a polymer that releases the drug(s) by diffusion through a polymer, or by flux through pores or by rupture of a polymer matrix. The drug delivery polymeric dosage form comprises the active compound contained in or on the polymer. The dosage form comprises at least one exposed surface at the beginning of dose delivery. The non-exposed surface, when present, can be coated with a pharmaceutically acceptable material impermeable to the passage of a drug. The dosage form may be manufactured by procedures known in the art, for example by blending a pharmaceutically acceptable carrier like polyethylene glycol, with a pre-determined dose of the active compound(s) at an elevated temperature (e.g., 37° C.), and adding it to a silastic medical grade elastomer with a cross-linking agent, for example, octanoate, followed by casting in a mold. The step is repeated for each optional successive layer. The system is allowed to set for 1 hour, to provide the dosage form. Representative polymers for manufacturing such sustained release dosage forms include, but are not limited to, olefin, and vinyl polymers, addition polymers, condensation polymers, carbohydrate polymers, and silicon polymers as represented by polyethylene, polypropylene, polyvinyl acetate, polymethylacrylate, polyisobutylmethacrylate, poly alginate, polyamide and polysilicon. These polymers and procedures for manufacturing them have been described in the art (see, e.g., Coleman et al., Polymers 1990, 31, 1187-1231; Roerdink et al., Drug Carrier Systems 1989, 9, 57-10; Leong et al., Adv. Drug Delivery Rev. 1987, 1, 199-233; and Roff et al., Handbook of Common Polymers 1971, CRC Press; U.S. Pat. No. 3,992,518).

In other embodiments of the invention, the compositions and dosage forms comprise an active therapeutic agent incorporated with or contained in beads that on dissolution or diffusion release the active compound over an extended period of hours, for example over a period of at least 6 hours, over a period of at least 8 hours, over a period of at least 12 hours, or over a period of up to 24 hours or longer. The drug-releasing beads may have a central composition or core comprising an active compound of Formula I and a pharmaceutically acceptable carrier, along with one or more optional excipients such as a lubricants, antioxidants, dispersants, and buffers. The beads may be medical preparations with a diameter of about 1 to 2 mm. In exemplary embodiments they are formed of non-cross-linked materials to enhance their discharge from the gastrointestinal tract. The beads may be coated with a release rate-controlling polymer that gives a timed release pharmacokinetic profile. In alternate embodiments the beads may be manufactured into a tablet for therapeutically effective drug administration. The beads can be made into matrix tablets by direct compression of a plurality of beads coated with, for example, an acrylic resin and blended with excipients such as hydroxypropylmethyl cellulose. The manufacture and processing of beads for use within the invention is described in the art (see, e.g., Lu, Int. J. Pharm., 1994, 112, 117-124; Pharmaceutical Sciences by Remington, 14.sup.th ed, pp 1626-1628 (1970); Fincher, J. Pharm. Sci. 1968, 57, 1825-18.35; and U.S. Pat. No. 4,083,949, each incorporated by reference) as has the manufacture of tablets (Pharmaceutical Sciences, by Remington, 17^{th} Ed, Ch. 90, pp 1603-1625, 1985, incorporated herein by reference).

In another embodiment of the invention, the dosage form comprises a plurality of tiny pills or mini-tablets. The tiny pills or mini-tablets provide a number of individual doses for providing various time doses for achieving a sustained-release drug delivery profile over an extended period of time up to 24 hours. The tiny pills or mini-tablets may comprise a hydrophilic polymer selected from the group consisting of a polysaccharide, agar, agarose, natural gum, alkali alginate including sodium alginate, carrageenan, fucoidan, furcellaran, laminaran, hypnea, gum arabic, gum ghatti, gum karaya, grum tragacanth, locust bean gum, pectin, amylopectin, gelatin, and a hydrophilic colloid. The hydrophilic polymer may be formed into a plurality (e.g., 4 to 50) tiny pills or mini-tablet, wherein each tiny pill or mini-tablet comprises a pre-determined dose of the active therapeutic agent, e.g., a dose of about 10 ng, 0.5 mg, 1 mg, 1.2 mg, 1.4 mg, 1.6 mg, 5.0 mg etc. The tiny pills and mini-tablets may further comprise a release rate-controlling wall of 0.001 up to 10 mm thickness to provide for timed release of the active compound. Representative wall forming materials include a triglyceryl ester selected from the group consisting of glyceryl tristearate, glyceryl monostearate, glyceryl dipalmitate, glyceryl laureate, glyceryl didecenoate and glyceryl tridenoate. Other wall forming materials comprise polyvinyl acetate, phthalate, methylcellulose phthalate and microporous olefins. Procedures for manufacturing tiny pills and mini-tablets are known in the art (see, e.g., U.S. Pat. Nos. 4,434,153; 4,721,613; 4,853,229; 2,996,431; 3,139,383 and 4,752,470, each incorporated herein by reference). The tiny pills and mini-tablets may further comprise a blend of particles, which may include particles of different sizes and/or release properties, and the particles may be contained in a hard gelatin or non-gelatin capsule or soft gelatin capsule.

In yet another embodiment of the invention, drug-releasing lipid matrices can be used to formulate therapeutic compositions and dosage forms comprising an active therapeutic agent. In one exemplary embodiment, solid microparticles of the active compound are coated with a thin controlled release layer of a lipid (e.g., glyceryl behenate and/or glyceryl palmitostearate) as disclosed in Farah et al., U.S. Pat. No. 6,375,987 and Joachim et al., U.S. Pat. No. 6,379,700 (each incorporated herein by reference). The lipid-coated particles can optionally be compressed to form a tablet. Another controlled release lipid-based matrix material which is suitable for use in the sustained release compositions and dosage forms of the invention comprises polyglycolized glycerides, e.g., as described in Roussin et al., U.S. Pat. No. 6,171,615 (incorporated herein by reference).

In other embodiments of the invention, drug-releasing waxes can be used for producing sustained release compositions and dosage forms comprising an active therapeutic agent. Examples of suitable sustained drug-releasing waxes include, but are not limited to, carnauba wax, candedilla wax, esparto wax, ouricury wax, hydrogenated vegetable oil, bees wax, paraffin, ozokerite, castor wax, and mixtures thereof (see, e.g., Cain et al. U.S. Pat. No. 3,402,240; Shtohryn et al. U.S. Pat. No. 4,820,523; and Walters, U.S. Pat. No. 4,421,736, each incorporated herein by reference).

In still another embodiment, osmotic delivery systems are used for sustained release delivery of an active therapeutic agent (see, e.g., Verma et al., Drug Dev. Ind. Pharm., 2000, 26:695-708, incorporated herein by reference). In one exemplary embodiment, the osmotic delivery system is an OROS® system (Alza Corporation, Mountain View, Calif.) and is adapted for oral sustained release delivery of drugs (see, e.g., U.S. Pat. No. 3,845,770; and U.S. Pat. No. 3,916,899, each incorporated herein by reference).

In another embodiment of the invention, the dosage form comprises an osmotic dosage form, which comprises a semi-permeable wall that surrounds a therapeutic composition comprising the active therapeutic agent. In use within a patient, the osmotic dosage form comprising a homogenous composition imbibes fluid through the semipermeable wall into the dosage form in response to the concentration gradient across the semipermeable wall. The therapeutic composition in the dosage form develops osmotic energy that causes the therapeutic composition to be administered through an exit from the dosage form over a prolonged period of time up to 24 hours (or even in some cases up to 30 hours) to provide controlled and sustained prodrug release. These delivery platforms can provide an essentially zero order delivery profile as opposed to the spiked profiles of immediate release formulations.

In alternate embodiments of the invention, the dosage form comprises another osmotic dosage form comprising a wall surrounding a compartment, the wall comprising a semipermeable polymeric composition permeable to the passage of fluid and substantially impermeable to the passage of the active compound present in the compartment, a drug-containing layer composition in the compartment, a hydrogel push layer composition in the compartment comprising an osmotic formulation for imbibing and absorbing fluid for expanding in size for pushing the active therapeutic agent composition layer from the dosage form, and at least one passageway in the wall for releasing the drug composition. This osmotic system delivers the active compound by imbibing fluid through the semipermeable wall at a fluid imbibing rate determined by the permeability of the semipermeable wall and the osmotic pressure across the semipermeable wall causing the push layer to expand, thereby delivering the active compound through the exit passageway to a patient over a prolonged period of time (up to 24 or even 30 hours). The hydrogel layer composition may comprise 10 mg to 1000 mg of a hydrogel such as a member selected from the group consisting of a polyalkylene oxide of 1,000,000 to 8,000,000 which are selected from the group consisting of a polyethylene oxide of 1,000,000 weight-average molecular weight, a polyethylene oxide of 2,000,000 molecular weight, a polyethylene oxide of 4,000,000 molecular weight, a polyethylene oxide of 5,000,000 molecular weight, a polyethylene oxide of 7,000,000 molecular weight and a polypropylene oxide of the 1,000,000 to 8,000,000 weight-average molecular weight; or 10 mg to 1000 mg of an alkali carboxymethylcellulose of 10,000 to 6,000,000 weight average molecular weight, such as sodium carboxymethylcellulose or potassium carboxymethylcellulose. The hydrogel expansion layer may comprise a hydroxyalkylcellulose of 7,500 to 4,500,00 weight-average molecular weight (e.g., hydroxymethylcellulose, hydroxyethylcellulose, hydroxypropylcellulose, hydroxybutylcellulose or hydroxypentylcellulose), an osmagent, e.g., selected from the group consisting of sodium chloride, potassium chloride, potassium acid phosphate, tartaric acid, citric acid, raffinose, magnesium sulfate, magnesium chloride, urea, inositol, sucrose, glucose and sorbitol, and other agents such a hydroxypropylalkylcellulose of 9,000 to 225,000 average-number molecular weight (e.g., hydroxypropylethylcellulose, hydroxypropypentylcellulose, hydroxypropylmethylcellulose, or hydropropylbutylcellulose), ferric oxide, antioxidants (e.g., ascorbic acid, butylated hydroxyanisole, butylatedhydroxyquinone, butylhydroxyanisol, hydroxycomarin, butylated hydroxytoluene, cephalm, ethyl gallate, propyl gallate, octyl gallate, lauryl gallate, propyl-hydroxybenzoate, trihydroxybutylrophenone, dimethylphenol, dibutylphenol, vitamin E, lecithin and ethanolamine), and/or lubricants (e.g., calcium stearate, magnesium stearate, zinc stearate, magnesium oleate, calcium palmitate, sodium suberate, potassium laureate, salts of fatty acids, salts of alicyclic acids, salts of aromatic acids, stearic acid, oleic acid, palmitic acid, a mixture of a salt of a fatty, alicyclic or aromatic acid, and a fatty, alicyclic, or aromatic acid).

In the osmotic dosage forms, the semipermeable wall comprises a composition that is permeable to the passage of fluid and impermeable to passage of the active therapeutic agent. The wall is nontoxic and comprises a polymer selected from the group consisting of a cellulose acylate, cellulose diacylate, cellulose triacylate, cellulose acetate, cellulose diacetate and cellulose triacetate. The wall typically comprises 75 wt % (weight percent) to 100 wt % of the cellulosic wall-forming polymer; or, the wall can comprise additionally 0.01 wt % to 80 wt % of polyethylene glycol, or 1 wt % to 25 wt % of a cellulose ether (e.g., hydroxypropylcellulose or a hydroxypropylalkycellulose such as hydroxypropylmethylcellulose). The total weight percent of all components comprising the wall is equal to 100 wt %. The internal compartment comprises the drug-containing composition alone or in layered position with an expandable hydrogel composition. The expandable hydrogel composition in the compartment increases in dimension by imbibing the fluid through the semipermeable wall, causing the hydrogel to expand and occupy space in the compartment, whereby the drug composition is pushed from the dosage form. The therapeutic layer and the expandable layer act together during the operation of the dosage form for the release of drug to a patient over time. The dosage form comprises a passageway in the wall that connects the exterior of the dosage form with the internal compartment. The osmotic powered dosage form delivers the active compound of active therapeutic agent from the dosage form to the patient at a zero order rate of release over a period of up to about 24 hours. As used herein, the expression “passageway” comprises means and methods suitable for the metered release of an active therapeutic agent from the compartment of an osmotic dosage form. The exit means comprises at least one passageway, including orifice, bore, aperture, pore, porous element, hollow fiber, capillary tube, channel, porous overlay, or porous element that provides for the osmotic controlled release of the active compound. The passageway includes a material that erodes or is leached from the wall in a fluid environment of use to produce at least one controlled-release dimensioned passageway. Representative materials suitable for forming a passageway, or a multiplicity of passageways comprise a leachable poly(glycolic) acid or poly(lactic) acid polymer in the wall, a gelatinous filament, poly(vinyl alcohol), leach-able polysaccharides, salts, and oxides. A pore passageway, or more than one pore passageway, can be formed by leaching a leachable compound, such as sorbitol, from the wall. The passageway possesses controlled-release dimensions, such as round, triangular, square and elliptical, for the metered release of prodrug from the dosage form. The dosage form can be constructed with one or more passageways in spaced apart relationship on a single surface or on more than one surface of the wall. The expression “fluid environment” denotes an aqueous or biological fluid as in a human patient, including the gastrointestinal tract. Passageways and equipment for forming passageways are disclosed in U.S. Pat. Nos. 3,845,770; 3,916,899; 4,063,064; 4,088,864; 4,816,263; 4,200,098; and 4,285,987 (each incorporated herein by reference).

Within other aspects of the invention, microparticle, microcapsule, and/or microsphere drug delivery technologies can be employed to provide sustained release delivery of an active therapeutic agent within the compositions, dosage forms and methods of the invention. A variety of methods is known by which an active compound of Formula I can be encapsulated in the form of microparticles, for example using by encapsulating the active compound within a biocompatible, biodegradable wall-forming material (e.g., a polymer)—to provide sustained or delayed release of the active compound. In these methods, the active compound is typically dissolved, dispersed, or emulsified in a solvent containing the wall forming material. Solvent is then removed from the microparticles to form the finished microparticle product. Examples of conventional microencapsulation processes are disclosed, e.g., in U.S. Pat. Nos. 3,737,337; 4,389,330; 4,652,441; 4,917,893; 4,677,191; 4,728,721; 5,407,609; 5,650,173; 5,654,008; and 6,544,559 (each incorporated herein by reference). These documents disclose methods that can be readily implemented to prepare microparticles containing an active therapeutic agent in a sustained release formulation according to the invention. As explained, for example, in U.S. Pat. No. 5,650,173, by appropriately selecting the polymeric materials, a microparticle formulation can be made in which the resulting microparticles exhibit both diffusional release and biodegradation release properties. For a diffusional mechanism of release, the active agent is released from the microparticles prior to substantial degradation of the polymer. The active agent can also be released from the microparticles as the polymeric excipient erodes. In addition, U.S. Pat. No. 6,596,316 (incorporated herein by reference) discloses methods for preparing microparticles having a selected release profile for fine tuning a release profile of an active agent from the microparticles.

In another embodiment of the invention, enteric-coated preparations can be used for oral sustained release administration. Preferred coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release). Enteric coatings may function as a means for mediating sustained release of the active compound of Formula I by providing one or more barrier layers, which may be located entirely surrounding the active compound, between layers of a multi-layer solid dosage form (see below), and/or on one or more outer surfaces of one or multiple layers of a multi-layer solid dosage form (e.g., on end faces of layers of a substantially cylindrical tablet). Such barrier layers may, for example, be composed of polymers which are either substantially or completely impermeable to water or aqueous media, or are slowly erodible in water or aqueous media or biological liquids and/or which swell in contact with water or aqueous media. Suitable polymers for use as a barrier layer include acrylates, methacrylates, copolymers of acrylic acid, celluloses and derivatives thereof such as ethylcelluloses, cellulose acetate propionate, polyethylenes and polyvinyl alcohols etc. Barrier layers comprising polymers which swell in contact with water or aqueous media may swell to such an extent that the swollen layer forms a relatively large swollen mass, the size of which delays its immediate discharge from the stomach into the intestine. The barrier layer may itself contain active material content, for example the barrier layer may be a slow or delayed release layer. Barrier layers may typically have an individual thickness of 10 microns up to 2 mm. Suitable polymers for barrier layers which are relatively impermeable to water include the Methocel™ series of polymers, used singly or combined, and Ethocel™ polymers. Such polymers may suitably be used in combination with a plasticizer such as hydrogenated castor oil. The barrier layer may also include conventional binders, fillers, lubricants and compression acids etc such as Polyvidon K30 (trade mark), magnesium stearate, and silicon dioxide.

Additional enteric coating materials for mediating sustained release of an active therapeutic agent include coatings in the form of polymeric membranes, which may be semipermeable, porous, or asymmetric membranes (see, e.g., U.S. Pat. No. 6,706,283, incorporated herein by reference). Coatings of these and other types for use within the invention may also comprise at least one delivery port, or pores, in the coating, e.g., formed by laser drilling or erosion of a plug of water-soluble material. Other useful coatings within the invention including coatings that rupture in an environment of use (e.g., a gastrointestinal compartment) to form a site of release or delivery port. Exemplary coatings within these and other embodiments of the invention include poly(acrylic) acids and esters; poly(methacrylic) acids and esters; copolymers of poly(acrylic) and poly(methacrylic) acids and esters; cellulose esters; cellulose ethers; and cellulose ester/ethers.

Additional coating materials for use in constructing solid dosage forms to mediate sustained release of an active therapeutic agent include, but are not limited to, polyethylene glycol, polypropylene glycol, copolymers of polyethylene glycol and polypropylene glycol, poly(vinylpyrrolidone), ethyl cellulose, hydroxyethyl cellulose, hydroxypropyl cellulose, carboxymethyl cellulose, carboxymethylethyl cellulose, starch, dextran, dextrin, chitosan, collagen, gelatin, bromelain, cellulose acetate, unplasticized cellulose acetate, plasticized cellulose acetate, reinforced cellulose acetate, cellulose acetate phthalate, cellulose acetate trimellitate, hydroxypropylmethylcellulose, hydroxypropylmethyl-cellulose phthalate, hydroxypropylmethylcellulose acetate succinate, hydroxypropylmethylcellulose acetate trimellitate, cellulose nitrate, cellulose diacetate, cellulose triacetate, agar acetate, amylose triacetate, beta glucan acetate, beta glucan triacetate, acetaldehyde dimethyl acetate, cellulose acetate ethyl carbamate, cellulose acetate phthalate, cellulose acetate methyl carbamate, cellulose acetate succinate, cellulose acetate dimethaminoacetate, cellulose acetate ethyl carbonate, cellulose acetate chloroacetate, cellulose acetate ethyl oxalate, cellulose acetate methyl sulfonate, cellulose acetate butyl sulfonate, cellulose acetate propionate, cellulose acetate p-toluene sulfonate, triacetate of locust gum bean, cellulose acetate with acetylated hydroxyethyl cellulose, hydroxylated ethylene-vinylacetate, cellulose acetate butyrate, polyalkenes, polyethers, polysulfones, polyethersulfones, polystyrenes, polyvinyl halides, polyvinyl esters and ethers, natural waxes and synthetic waxes.

In additional embodiments of the invention, sustained release of the psychotherapeutic therapeutic compound and/or α and/or β blocker is provided by formulating the active compound in a dosage form comprising a multi-layer tablet or other multi-layer or multi-component dosage form. In exemplary embodiments, the active compound is formulated in layered tablets, for example having a first layer which is an immediate release layer and a second layer which is a slow release layer. Other multi-layered dosage forms of the invention may comprise a plurality of layers of compressed active ingredient having variable (i.e., selectable) release properties selected from immediate, extended and/or delayed release mechanisms. Multi-layered tablet technologies useful to produce sustained release dosage forms of an active compound of Formula I are described, for example, in International Publications WO 95/20946; WO 94/06416; and WO 98/05305 (each incorporated herein by reference). Other multi-component dosage forms for providing sustained delivery of an active therapeutic agent include tablet formulations having a core containing the active compound coated with a release retarding agent and surrounded by an outer casing layer (optionally containing the active compound) (see, e.g., International Publication WO 95/28148, incorporated herein by reference). The release retarding agent is an enteric coating, so that there is an immediate release of the contents of the outer core, followed by a second phase from the core which is delayed until the core reaches the intestine. Additionally, International Publication WO 96/04908 (incorporated herein by reference) describes tablet formulations which comprise an active agent in a matrix, for immediate release, and granules in a delayed release form comprising the active agent. Such granules are coated with an enteric coating, so release is delayed until the granules reach the intestine. International Publication WO 96/04908 (incorporated herein by reference) describes delayed or sustained release formulations formed from granules which have a core comprising an active agent, surrounded by a layer comprising the active agent.

Another useful multi-component (bi-layer tablet) dosage form for sustained delivery of active compounds of the present invention is described in U.S. Pat. No. 6,878,386 (incorporated herein by reference). Briefly, the bilayer tablet comprises an immediate release and a slow release layer, optionally with a coating layer. The immediate release layer may be, for example, a layer which disintegrates immediately or rapidly and has a composition similar to that of known tablets which disintegrate immediately or rapidly. An alternative type of immediate release layer may be a swellable layer having a composition which incorporates polymeric materials which swell immediately and extensively in contact with water or aqueous media, to form a water permeable but relatively large swollen mass. Active material content may be immediately leached out of this mass. The slow release layer may have a composition comprising the active therapeutic agent with a release retarding vehicle, matrix, binder, coating, or excipient which allows for slow release of the active compound. Suitable release retarding excipients include pH sensitive polymers, for instance polymers based upon methacrylic acid copolymers, which may be used either alone or with a plasticiser; release-retarding polymers which have a high degree of swelling in contact with water or aqueous media such as the stomach contents; polymeric materials which form a gel on contact with water or aqueous media; and polymeric materials which have both swelling and gelling characteristics in contact with water or aqueous media. Release retarding polymers which have a high degree of swelling include, inter alia, cross-linked sodium carboxymethylcellulose, cross-linked hydroxypropylcellulose, high-molecular weight hydroxypropylmethylcellulose, carboxymethylamide, potassium methacrylatedivinylbenzene co-polymer, polymethylmethacrylate, cross-linked polyvinylpyrrolidone, high-molecular weight polyvinylalcohols etc. Release retarding gellable polymers include methylcellulose, carboxymethylcellulose, low-molecular weight hydroxypropylmethylcellulose, low-molecular weight polyvinylalcohols, polyoxyethyleneglycols, non-cross linked polyvinylpyrrolidone, xanthan gum etc. Release retarding polymers simultaneously possessing swelling and gelling properties include medium-viscosity hydroxypropylmethylcellulose and medium-viscosity polyvinylalcohols. An exemplary release-retarding polymer is xanthan gum, in particular a fine mesh grade of xanthan gum, preferably pharmaceutical grade xanthan gum, 200 mesh, for instance the product Xantural 75 (also known as Keltrol CR™ Monsanto, 800 N Lindbergh Blvd, St Louis, Mo. 63167, USA). Xanthan gum is a polysaccharide which upon hydration forms a viscous gel layer around the tablet through which the active has to diffuse. It has been shown that the smaller the particle size, the slower the release rate. In addition, the rate of release of active compound is dependent upon the amount of xanthan gum used and can be adjusted to give the desired profile. Examples of other polymers which may be used within these aspects of the invention include Methocel K4M™, Methocel E5™, Methocel E5O™, Methocel E4M™, Methocel K15M™ and Methocel K100M™. Other known release-retarding polymers which may be incorporated within this and other embodiments of the invention to provide a sustained release composition or dosage form of an active therapeutic agent include, hydrocolloids such as natural or synthetic gums, cellulose derivatives other than those listed above, carbohydrate-based substances such as acacia, gum tragacanth, locust bean gum, guar gum, agar, pectin, carageenin, soluble and insoluble alginates, carboxypolymethylene, casein, zein, and the like, and proteinaceous substances such as gelatin.

Within other embodiments of the invention, a sustained release delivery device or system is placed in the subject in proximity of the target of the active compound, thus requiring only a fraction of the systemic dose (see, e.g., Goodson, in “Medical Applications of Controlled Release,” supra, vol. 2, pp. 115-138, 1984; and Langer, 1990, Science 249:1527-1533, each incorporated herein by reference). En other embodiments, an oral sustained release pump may be used (see, e.g., Langer, supra; Sefton, 1987, CRC Crit. Ref. Biomed. Eng. 14:201; and Saudek et al., 1989, N. Engl. J. Med. 321:574, each incorporated herein by reference).

The pharmaceutical compositions and dosage forms of the current invention will typically be provided for administration in a sterile or readily sterilizable, biologically inert, and easily administered form.

In other embodiments the invention provides pharmaceutical kits for reducing, treating, preventing or alleviating symptoms in a human subject suffering from or at risk for an anxiety disorder. The kits comprise an active therapeutic agent in an effective amount, and a container means for containing the active therapeutic agent for coordinate administration to the said subject (for example a container, divided bottle, or divided foil pack). The container means can include a package bearing a label or insert that provides instructions for multiple uses of the kit contents to treat the anxiety disorder and reduce symptoms in the subject. In more detailed embodiments, the active therapeutic agent are admixed or co-formulated in a single, combined dosage form, for example a liquid or solid oral dosage form. In alternate embodiments, the active therapeutic agent are contained in the kit in separate dosage forms for coordinate administration. An example of such a kit is a so-called blister pack. Blister packs are well-known in the packaging industry and are widely used for the packaging of pharmaceutical dosage forms (tablets, capsules and the like). Blister packs generally consist of a sheet of relatively stiff material covered with a foil of a transparent material. During the packaging process, recesses are formed in the plastic foil. The recesses have the size and shape of the tablets or capsules to be packed. Next, the tablets or capsules are placed in the recesses and the sheet of relatively stiff material is sealed against the plastic foil at the face of the foil that is opposite from the direction in which the recesses were formed. As a result, the tablets or capsules are sealed in the recesses between the plastic foil and the sheet. Preferably, the strength of the sheet is such that the tablets or capsules can be removed from the blister pack by manually applying pressure on the recesses whereby an opening is formed in the sheet at the place of the recess. The tablet or capsule can then be removed from the opening. Often, a memory aid is provided on the kit, such as in the form of numbers next to the tablets or capsules whereby the numbers correspond with the days of the regimen which the tablets or capsules so specified should be ingested. Other variations of memory aids will be readily apparent.

The following examples illustrate certain aspects of the invention, but are not intended to limit in any manner the scope of the invention.

### Example 1

### Animal Model for Testing the Efficacy of Carvedilol on Avoidance Behavior

Subjects are male, Sprague-Dawley (SD) rats. They are approximately 60-days-old and 300-350 g at the time of testing. They are maintained on ad lib food and water except during the stress and escape/avoidance sessions. Subjects are maintained on a 12:12 light/dark cycle, with lights on at 0700. (Brennan et al., 2005)

All animals are randomly assigned to groups: a control group which does not receive shocks, unmedicated animals that receive shocks, animals that receive carvedilol and do not receive shocks, animals that receive carvedilol and receive shocks. The animals that receive shocks are restrained in plastic tubes (Harvard Apparatus, Inc, Holliston, Mass., USA) and have tail electrodes attached. The single shock session consists of forty 3-s, 2-mA tailshocks, presented on a variable time (VT) 3-min schedule, making the shock session approximately 2 h in duration.

Escape/avoidance sessions are conducted in 4 operant chambers (Coulbourn, Inc, Allentown, Pa., USA). The chambers are 30.5 cm wide×25.4 cm deep×30.5 cm high and have a lever mounted on one wall. Subjects are allowed approximately 1 min to explore the chamber before the session begins.

The trial begins with a warning signal and houselight. If the animal does not make a leverpress after 60 s of the warning signal, they receive 1.0-mA footshock through the grid floor. The shock, warning signal, and houselight are all terminated by a leverpress. A leverpress after the shock is classified as an “escape”, while a response that occurred during the initial 60 s of the warning signal before the shock is classified as an “avoidance.” The number of escape and avoidance responses by hour across the session, percent avoidance, and the number of leverpresses during the safety period (a putative measure of anxiety) are analyzed. Prior exposure to tailshock without medication leads to an increase in the number of avoidance responses 24 h later. Prior exposure to tailshock with medication will lead to a decrease in the number of avoidance response 24 h later compared to those animals that did not receive medication.

### Example 2

### Animal Testing for the Efficacy of Carvedilol on Protecting Brain Tissue from Damage During Stress

Male Fischer 344 rats, 3 months old are used. Four 200-mg corticosterone SR pellets (Innovative Research of America, Toledo, Ohio), which released over 90 days, or placebo pellets are implanted subcutaneously 4 cm lateral of the median line under general anesthesia in each rat. Carvedilol pellets or placebo pellets were implanted at the nape of the neck in each rat every 3 weeks (four times). (Levy et al., 2001)

Rats are randomly assigned into two treatment groups: (1) corticosterone-treated (four SR pellets to each rat) or (2) placebo-treated (same pellets without drug). Each of the above groups was further subdivided into (a) carvedilol-treated (four consecutive implantations every 3 weeks) or (b) placebo-treated (same pellets without drug).

Four weeks following termination of the corticosterone/carvedilol treatments, rats participate for 6 days in a radial-armmaze (RAM) learning test. Time to complete the task and number of errors is recorded.

Rats are then analyzed for histology. Rats are anesthetized and perfused (saline followed by a formaldehyde mixture). Brains are fixed, dehydrated, and paraffin embedded. Six-micrometer coronal sections, at the hippocampal level, are stained with hematoxylin and eosin. Quantitative analysis of morphological changes is carried out by counting the number of damaged cells, as well as the total number of cells, in various hippocampal regions.

During the 90 days of treatment, corticosterone levels in the two corticosterone-treated groups is kept in the range of 150 to 350 ng/ml, corresponding to the range found normally in rats under mild stress. The two control groups are shown to have normal corticosterone levels. Under these conditions, the corticosterone without carvedilol group exhibit morphological changes in district hippocampal areas. Carvedilol, administered concomitantly, will provide protection against the hormonal-induced brain damage. In the behavioral tests, the cognitive parameter (number of correct entries out of the first eight in the RAM) improves similarly during the 6 training days in all four groups. A statistically significant difference will be found between groups not treated with carvedilol.

### Example 3

### Effect of Carvedilol on Receptors in Rats Subject to Single Prolonged Stress

Male Wistar rats (150 g-200 g) are used for all experiments. Rats are housed under temperature-controlled (22±1° C.) conditions, and maintained at 12:12 light/dark cycle (lights on at 07:00 and off at 19:00) with free access to food and water. (Zhe et al., 2008)

The animals are divided into four groups: 1) control group; 2) stressed group without medication; 3) stressed group administered carvedilol prior to being stressed; 4) stressed group administered carvedilol after being stressed.

Control animals remain in their home cages with no handling for 7 days and are killed at the same time as stressed groups. Stressed-rats are given carvedilol prior to or after receiving a stressing procedure on the first day. The single session of prolonged stress consists of: restraint for 2 hr, followed by forced swim for 20 min (24° C.), followed by ether anesthesia. They are then allowed to remain in their home cages without interference and are killed 7 days later.

The rats are anaesthetized with 50 mg/kg body weight sodium pentobarbital, and the brains are removed from the skull after perfusion fixation with 4% paraformaldehyde (PFA) in 0.1 M phosphate buffer saline (PBS). The brains are then quickly frozen using powdered dry ice and cut into 12 μm thick frontal sections on a cryostat. After drying the sections are stored at −20° C. before immunohistochemistry.

The sections are treated with 5% bovine serum albumin (BSA), 0.3% Triton X-100 in PBS for 30 min at room temperature (RT) to block non-specific staining. The sections are then incubated with rabbit polyclonal antibody against MR (Santa Cruz, Calif., USA; 1:300) or rabbit polyclonal antibody against GR (Santa Cruz; 1:1,000) in 2% BSA-PBS for 24 hr at 4° C. After being washed with PBS, the sections are incubated with biotinylated-goat anti anti-rabbit IgG (Boster, China; 1:100) for 2 hr and then with streptoavidin-biotin peroxidase complex (SABC) for 1 hr. The sections are washed four times with PBS after each of incubation and subsequently are incubated with 3,3′-diaminobenzidine (DAB) and H₂O₂. To assess nonspecific staining, a few sections in every experiment are incubated in buffer without primary antibody.

Some samples are subject to Western blotting. Freshly frozen hippocampus of control rats and stressed rats respectively are homogenized with sample buffer containing 200 mM Tris-buffered saline, pH 7.5 (TBS), 4% sodium dodecyl sulfate (SDS), 20% glycerol, 10% 2-mercaptoethanol and are denatured by boiling for 3 min. Sample (50 μg/lane) are loaded on a 7.5% SDS-polyacrylamide gel (PAGE), and electroblotted onto a polyvinylidene difluoride (PVDF) membrane (Millipore Corp., Bedford, Mass., USA) from the gel by a semi-dry blotting apparatus (Bio-Rad Laboratories, Inc, Hercules, Calif., USA). The blotted membrane are then blocked with 1.5% skim milk, 0.05% Tween-20 in TBS (TBST) at 4° C. overnight and then incubated with rabbit polyclonal antibody against MR (Santa Cruz, USA; 1:300) or rabbit polyclonal antibody against GR (Santa Cruz, USA; 1:500) at 4° C. for 24 hr. Blots are washed three times with TBST, and then incubated with horseradish peroxidase-conjugated goat anti-rabbit IgG second antibody (Bio-Rad Laboratories, Inc, USA; 1:2,000) for 2 hr at room temperature. After the incubation, blots are washed three times with TBST before visualization by enhanced chemiluminescence (ECL; Amersham Pharmacia Biotech, Buckinghamshire, UK). To confirm equal protein loading the same blots are re-incubated with antibodies specific for β-actin (Abeam, British; 1:1,000). Immunoreaction for β-actin is detected with the ECL.

In the stressed group that did not receive carvedilol, stressing induced down regulation of mineralocorticoid receptor (MR) and glucocorticoid receptor (GR) protein expression and the change in MR/GR ratio in the hippocampus. Stressing induces different degrees of regulation in GR-ir distribution in the hippocampus by immunochemistry. Mice that are treated with carvedilol both prior to and following stress will show less down regulation in both MR and GR protein expression.

### Example 4

### Efficacy of Carvedilol Versus Placebo in the Treatment of Post Traumatic Stress Disorder

The efficacy of carvedilol was examined in a clinical trial using a parallel group, randomized, double-blind method with 50% of the outpatients receiving a flexible dose of alpha and beta blocker carvedilol for treatment of PTSD and the other 50% receiving a flexible close of matching placebo tablets. The trial consisted of a one week screening period followed by baseline symptom measurement and five weeks of PTSD pharmacotherapy. Patient assessments took place each week throughout the course of the trial.

Patients between 18 and 70 years of age were eligible for enrollment. A minimum score of 60 was required on the Clinician Administered PTSD Scale (CAPS-II) at the screening visit and all patients met clinical criteria for PTSD in accordance with the Diagnostic and Statistical Manual IV and confirmed by the Mini International Neuropsychological Interview (MINI).

Patients with a medical history of any of the following conditions were excluded: thyroid problems, tumor or other conditions that predispose the patient to a risk of seizure, bronchial asthma or related bronchospastic conditions, AV block, sick sinus syndrome, bradycardia or peripheral heart disease. Patients with current psychiatric diagnosis of bipolar disorder, schizophrenia, dementia or psychosis were also excluded. Any abnormal clinical laboratory or ECG test results judged clinically significant by the Principal Investigator resulted in exclusion. Females with a positive serum hCG pregnancy test or who were pregnant or lactating were excluded, and females of childbearing potential were required to use medically approved birth control methods for the duration of study and one month following the last dose.

Eighty-three subjects were enrolled and randomized to carvedilol or placebo. Six patients did not return after the baseline visit and were excluded from the analysis. The final efficacy analysis included 38 patients allocated to carvedilol, and 39 patients allocated to placebo. All of the patients, study coordinators, and principal investigators were blinded from the patient treatment allocation. Patients were randomly assigned to kits containing carvedilol or placebo at the baseline visit.

At the baseline visit, all patients were titrated to one dose of 3.125 mg carvedilol or placebo in the morning and an additional dose in the evening for the first week of the trial (6.25 mg carvedilol per day). At the discretion of the investigator the dose of carvedilol or placebo could be increased to a maximum of two tablets (6.250 mg carvedilol) in the morning and two tablets (6.250 mg carvedilol) in the evening during the following visit (week 1). At the week 2 visit the investigator could increase the dose of carvedilol or placebo to a maximum of 5 pills daily (15.625 mg carvedilol per day) based on patient tolerability and treatment response. After the second visit investigators were encouraged to maintain patient dose throughout the remainder of the five week trial although they were allowed to decrease the dose in the event that tolerability issues arose.

Based on data from previously conducted PTSD trials, the treatment effect of carvedilol for PTSD was considered to likely be in the medium range (Cohen's d=0.45). Given the possibility of this moderate treatment effect, a total sample size of 80 would provide statistical power of 80% to detect a treatment effect. In the event that a patient did not return after randomization, additional randomization slots were made available in effort to maintain the level of 80% power.

Although a minimum score of 60 on the CAPS-II was required at the screening visit as entry criteria, the Davidson Trauma Scale (DTS) was used as primary outcome measurement. The DTS is a 34-item patient rated scale with a maximum total score of 136. The DTS is designed to measure frequency and severity of three subtypes of PTSD symptoms: intrusion, avoidance and hyperarousal. Patients completed the DTS at the baseline visit and at each visit throughout the course of the trial.

Secondary outcome measures included the Insomnia Severity Index (ISI), the Clinician Global Impressions (CGI) Scale, and each of the three DTS subscales. The ISI is a self-rated 7-item scale (maximum score=28) designed to measure insomnia during clinical research. The ISI was completed at each visit throughout the trial. The CGI was also completed during each visit. As an additional secondary outcome, the change in CAPS-II score was evaluated from the screening visit to the last trial visit for patients that completed the trial.

The trial data analysis was designed to investigate the hypothesis that carvedilol would provide relief from symptoms for patients clinically diagnosed with PTSD. To evaluate this possibility, an analysis of covariance (ANCOVA) test was conducted on the primary outcome variable and a series of independent samples t tests were conducted on secondary outcome variables.

Each statistical test was conducted using SPSS version 19.0 (IBM). All patients that completed the baseline visit and at least one follow up measurement were included in the analysis. In the event that a patient did not complete the trial, the last observation carried forward (LOCF) method of efficacy analysis was used. Therefore, the final completed measurement of PTSD symptoms was used as the end data point in the event that patients terminated from the trial early.

In evaluating the primary efficacy outcome (change in DTS score), the ANCOVA was designed to factor in the severity of PTSD at the baseline rating. The final visit (week 5) DTS score was entered as the dependent variable and the baseline visit DTS score was entered as a covariate. Treatment assignment of the patients (drug=1, placebo=2) was entered as a categorical independent variable.

In evaluating outcome on secondary measures, the change in ISI, CGI and DTS subscale scores from baseline to the end of treatment visit (Baseline score—Week 5 score) was first calculated. During these analyses, an Independent Samples/Test was conducted for each of the secondary endpoints (change in ISI, CGI, DTS intrusion, avoidance and hyperarousal subscale scores). The change in scale/subscale score was entered as the dependent variable during each individual analysis and the treatment assignment of the patients (drug=1, placebo=2) was entered as a categorical independent variable.

For change in CAP-II scale score, a similar Independent Samples t Test was conducted using a completer only analysis. For this analysis, the dependent variable was change in CAPS-II score calculated as the score during screening visit less the score at final visit.

Besides the calculation of F mean comparisons, there possibly were other factors that could influence treatment response during PTSD trials. In this context, a series of post hoc analyses were conducted to evaluate the role of demographic factors of the patients such as sex, age, Body Mass Index (BMI) and veteran status. Additionally, the duration of episode or symptom subtypes of PTSD was evaluated for potential influence on treatment outcome.

Characteristics of the 83 patients that comprised the intent to treat sample are shown as Table 2. There were no significant differences in demographic or PTSD severity characteristics between the two groups at the baseline visit. The mean baseline DTS score for the entire sample was 91.9 and the mean ISI score for the entire sample was 19.9. The majority of patients entered the trial with a PTSD episode over five years in duration. Approximately 50% of the patients used concomitant medications throughout the trial.

TABLE 2
Characteristics of Patients with Post-traumatic Stress Disorder
Assigned to Carvedilol or Placebo

| | Carvedilol | | |
|---|---|---|---|
| | (n = 42) | Placebo (n = 41) | p Value |
| Mean Age | 40.0 | 41.6 | 0.59 |
| % Female | 59.5 | 43.9 | 0.15 |
| % Caucasian | 61.9 | 73.2 | 0.27 |
| % Military Veteran | 16.7 | 26.8 | 0.26 |
| % with PTSD episode duration | 81.0 | 70.7 | 0.28 |
| >5 years | | | |
| Mean Baseline DTS Score | 91.2 | 94.2 | 0.53 |
| Mean Baseline ISI Score | 19.7 | 20.6 | 0.35 |
| % Taking Concomitant | 59.5 | 46.3 | 0.31 |
| Medication(s) | | | |

Intent to Treat Sample
DTS = Davidson Trauma Scale
ISI = Insomnia Severity Index
Probability values were determined by Independent Samples t Tests (mean comparisons) or Chi-Square Test (comparison of proportions).

The majority of the patients randomized to carvedilol (28/38, 74%) were titrated to the maximum dosage allowed in the protocol (five 3.125 mg tablets daily). There were 5 patients titrated to 4 tablets per day, 2 patients were titrated to three tablets per day, and 3 patients were maintained on the minimum dose of two tablets. In the placebo group, 20/39 (51%) of the patients were titrated to the maximum dose of five tablets per day, 12 patients were titrated to 4 tablets per day, 5 patients were titrated to 3 tablets per day and 2 patients were maintained on the minimum dose of two tablets.

The ANCOVA that evaluated difference in visit 7 DTS scores between patients assigned to carvedilol versus placebo did not show a significant difference between the two groups, F(df=2.74)=0.98, p=0.33. The mean DTS change score for patients assigned to carvedilol was −24.6 (SD±23.8), and mean DTS change score for patients assigned to placebo was −19.2 (SD±31.1). Severity of PTSD was a significant predictor of visit 7 DTS total scores with patients having a higher score at baseline also having higher scores at the end of treatment, F(2.74)=30.42, p<0.001.

Analysis of the secondary outcome variables showed a similar trend with those assigned to carvedilol having some additional symptom relief although there were no significant differences between carvedilol and placebo groups for any of the secondary analyses

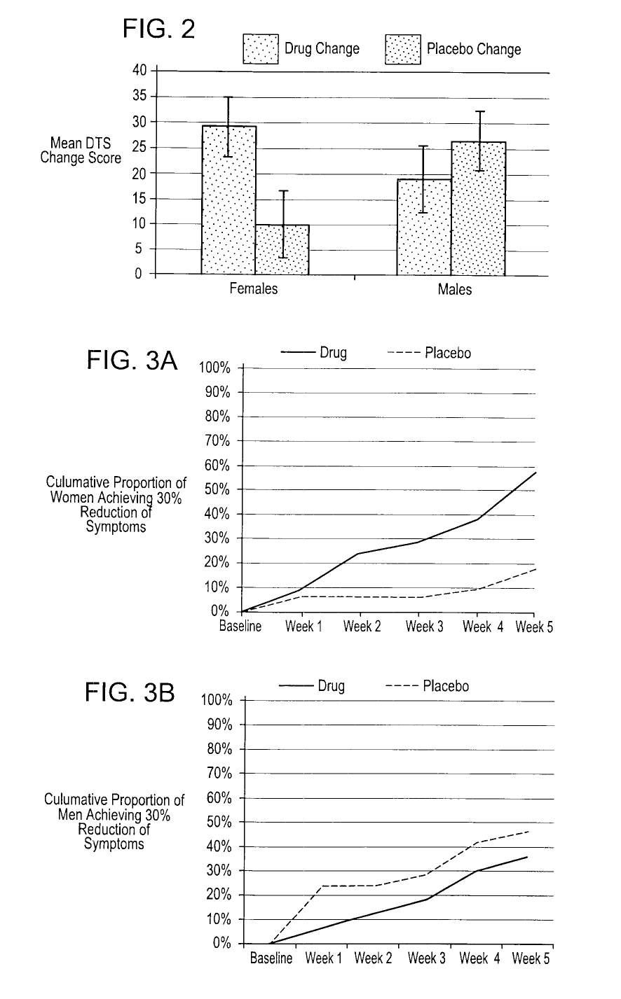

An additional ANOVA was conducted to evaluate any roll that sex of the patient may have on outcome of PTSD treatment with carvedilol or placebo. As shown in Table 3 and FIG. 2 there was a significant interaction between sex of the patient and overall outcome of treatment, F(df=72)=4.26, p=0.043. The mean change score for women assigned to carvedilol (n=21) was −29.2 (SD±26.5) and mean change score for women assigned to placebo (n=17) was −10.0±22.7. The mean change score for men assigned to carvedilol (n=17) was −18.8 (SD±19.1), and mean change score for men (n=22) assigned to placebo was −26.3 (SD±35.1). Change scores for men and women from both treatment arms are shown as FIG. 2.

TABLE 3
Results from Analysis of Covariance Conducted During Post Hoc Analysis
to Evaluate Role of Sex in Response to Carvedilol and Placebo for
PTSD Symptoms

| | Type III | | | | |
|---|---|---|---|---|---|
| | Sum of | Degrees of | Mean | | |
| Source | Squares | Freedom | Square | F | p value |
| Corrected Model | 28,108 | 4 | 7,027 | 9.58 | <0.001 |
| Intercept | 66.3 | 1 | 66.3 | 0.09 | 0.765 |
| V2DTS | 24,044 | 1 | 24,044 | 32.77 | <0.001 |
| Carvedilol vs | 833 | 1 | 833 | 1.14 | 0.29 |
| Placebo | | | | | |
| Sex of Patients | 156.6 | 1 | 156.6 | 0.213 | 0.645 |
| Interaction of | 3124 | 1 | 3,124 | 4.26 | 0.043 |
| Drug by Sex | | | | | |
| Error | 52,822 | 72 | 733.6 | | |
| Total | 458,371 | 77 | | | |
| Corrected Total | 80,930 | 76 | | | |

Adjusted R Squared = 0.311

Based on the finding of a significant interaction between sex of the patients and treatment assignment, a series of analyses was conducted on outcome variables for men and women separately and found that women treated with carvedilol had significantly better outcome than did women treated with placebo (see Table 4). This was not the case with men that enrolled in the trial as there were no significant differences between men treated with carvedilol as compared to placebo. Women that were assigned to carvedilol had significantly higher change scores on overall DTS scores (p=0.023), intrusiveness subscale scores (p=0.008) and CGI scores, (p=0.002).

TABLE 4
Comparisons of Change Scores on Primary and Secondary Outcome
Measures between Carvedilol and Placebo Based on Sex.

| | Change Scores for | | | Change Scores for | | |
|---|---|---|---|---|---|---|
| | Women | | | Men | | |
| Outcome | Carvedilol | Placebo | | Carvedilol | Placebo | |
| Measure | (n = 21) | (n = 17) | p value | (n = 17) | (n = 22) | p value |
| Davidson | −29.2 ± 26.5 | −10.0 ± 22.7 | 0.023 | −18.8 ± 19.1 | −26.3 ± 35.1 | 0.44 |
| Trauma Scale | | | | | | |
| (DTS) | | | | | | |
| DTS | −9.9 ± 8.2 | −2.2 ± 8.5 | 0.008 | −5.9 ± 7.5 | −4.0 ± 12.0 | 0.58 |
| Intrusiveness | | | | | | |
| Subscale | | | | | | |
| DTS Avoidance | −10.4 ± 14.0 | −3.3 ± 0.2 | 0.087 | −6.4 ± 8.3 | −12.6 ± 17.4 | 0.18 |
| Subscale | | | | | | |
| DTS | −9.0 ± 8.9 | −4.5 ± 7.1 | 0.099 | −6.6 ± 7.4 | −9.6 ± 10.0 | 0.30 |
| Hyperarousal | | | | | | |
| Subscale | | | | | | |
| Insomnia | −5.1 ± 6.3 | −2.1 ± 4.3 | 0.09 | −6.8 ± 6.7 | −4.9 ± 6.1 | 0.43 |
| Severity Index | | | | | | |
| (ISI) | | | | | | |
| Clinician's | −1.8 ± 0.2 | −0.5 ± 0.3 | 0.002 | −1.2 ± 1.3 | −1.3 ± 1.4 | 0.85 |
| Global | | | | | | |
| Impression | | | | | | |
| (CGI) | | | | | | |
| *Clinician | −21.7 ± 25.2 | −10.9 ± 20.5 | 0.195 | −16.2 ± 19.0 | −17.5 ± 17.5 | 0.84 |
| Administered | | | | | | |
| PTSD Scale | | | | | | |
| (CAPS II) | | | | | | |

Change scores for DTS, ISI and CGI were calculated from week 2 baseline visit to final visit using the last observation carried forward.
CAPS II change scores were calculated from week 1 screening visit to week 7 final visit using a completer only analysis.
Probability values were determined using Independent Samples t Tests with change scores as dependent variable and treatment (carvedilol or placebo) as an independent variable.

Time trends for the proportion of females assigned to carvedilol or placebo that did achieve a 30% reduction of symptoms is shown as FIG. 3 Part A. The difference in response trends between women assigned to carvedilol as compared to placebo was significant, Mantel-Cox χ²=5.78, p=0.016. The proportion of males assigned to carvedilol or placebo that did achieve a 30% reduction of symptoms is shown as FIG. 3 Part B. Although 25% of the men assigned to placebo responded during the first week, the time to response trends did not significantly differ between men assigned to carvedilol or placebo.

Based on these findings an additional analysis was conducted of any role that cause of PTSD may have on treatment outcome. For the subsample of women that experienced personal violation of physical or sexual assault the magnitude of treatment effect was particularly large. The mean change score for 15 female assault victims treated with carvedilol was −31.8 as compared to a change score of −7.8 for the 9 female assault victims assigned to placebo t (df=22)=2.2, p=0.041, for an effect size of d=0.95.

In view of the findings that carvedilol was effective for treatment of women patients with PTSD, an additional analysis was conducted to determine if experience as a military veteran might have a role on treatment outcome. Of the above-reported populations, all of the carvedilol-treated women (n=21) were not military veterans, and nearly all of the placebo-treated women (n=16; i.e., 16 of 17 total) were not military veterans. In contrast, 7 of the 17 men treated with carvedilol were veterans, and 10 of the 22 men treated with placebo were veterans. The PTSD scale change scores for the subpopulation that was inclusive of males and females that were not veterans are shown in Table 5.

TABLE 5
PTSD Scale Change Scores Inclusive of Males and Females that were
not Veterans, Mean Change (SD).

| | Carvedilol | Placebo | |
|---|---|---|---|
| Outcome | (n = 31, 69% | (n = 28, 57% | |
| Measure | female) | female) | t value |
| Davidson | −25.2 ± 25.3 | −15.2 ± 30.3 | t = 1.38, p = 0.17, |
| Trauma Scale | | | d = 0.36 |
| (DTS) | | | |
| DTS | −8.8 ± 8.8 | −3.1 ± 9.9 | t = 2.3, p = 0.02, |
| Intrusiveness | | | d = 0.61 |
| Subscale | | | |
| DTS Avoidance | −8.7 ± 12.4 | −5.8 ± 15.2 | t = 0.81, p = 0.42, |
| Subscale | | | d = 0.21 |
| DTS | −7.7 ± 6.3 | −6.3 ± 8.9 | t = 0.64, p = 0.52, |
| Hyperarousal | | | d = 0.18 |
| Subscale | | | |
| Insomnia | −5.8 ± 6.8 | −2.9 ± 5.5 | t = 1.8, p = 0.07, |
| Severity Index | | | d = 0.47 |
| (ISI) | | | |
| *Clinician | −20.6 ± 22.2 | −13.3 ± 20.1 | 0.195 |
| Administered | | | |
| PTSD Scale | | | |
| (CAPS II) | | | |

Change scores for DTS, and ISI were calculated from week 2 baseline visit to final visit using the last observation carried forward.
CAPS II change scores were calculated from week 1 screening visit to week 7 final visit using a completer only analysis.
Probability values were determined using Independent Samples t Tests with change scores as dependent variable and treatment (carvedilol or placebo) as an independent variable.

The results of this analysis indicate that carvedilol was effective for both men and women who were not military veterans, as the subjects treated with carvedilol had significantly better outcome than those treated with placebo.

Incidence of adverse events such as headaches, common cold and nausea were similar between the carvedilol and placebo groups. A total of 28 patients assigned to carvedilol (66%) reported adverse events and 26 patients assigned to placebo (63%) reported adverse events. The types of adverse events that occurred did not fall outside of the already established product guidelines for carvedilol. Adverse events that occurred in over 5% of carvedilol patients included somnolence, headache and common cold.

From the baseline visit to end of study the patients treated with carvedilol experienced a mean decrease in systolic blood pressure of −4.3±9.7 mm Hg that was significantly different from the mean increase in systolic blood pressure for patients assigned to placebo of +1.9±11.6 mm Hg, p=0.013. The mean change in in diastolic blood pressure (carvedilol=−0.7 mm Hg, placebo=+1.5 mm HG, p=0.122) and mean change in heart rate (carvedilol=−3.7 beats/minute, placebo=−1.6 beats/minute, p=0.292) were not significantly different between the two groups.

The results of this study demonstrated that carvedilol was effective for treatment of women patients with PTSD, and in particular, women that are victims of intimate violence such as physical or sexual assault. Moreover, the results indicated that carvedilol was effective for treatment of men and women patients with PTSD and who were not military veterans.

### Example 5

### Use of Carvedilol in Preventing Post Traumatic Stress Syndrome

Certain populations are more prone to the development of PTSD. For example, combat soldiers have an incidence rate of 25% and between 7 and 37% of firefighters will develop PTSD. Prevention of PTSD can therefore be determined by comparing treated populations of these groups to untreated populations. Subjects are identified who are between the ages of 18-70 years, inclusive, and are in jobs with a high incidence of PTSD such as soldiers, firefighters, policemen, emergency workers or other first responders. They will be excluded if they have current, unstable and significant medical conditions or illness including bronchial asthma or related bronchospastic conditions, AV block, sick sinus syndrome, bradycardia, or peripheral heart disease; any abnormal clinical laboratory test results or ECG results which are judged by the investigator as clinically significant; a history of unstable Thyroid disorder; history of seizure disorder, tumor or other CNS condition that predisposes the patient to risk of seizure; be pregnant; have a history or current diagnosis of bipolar, psychosis, schizophrenia or dementia; a primary diagnosis of depression. Depression, anxiety or another similar Axis 1 disorder secondary to PTSD diagnosis is acceptable. They must not be taking anti-psychotics, anti-depressants, anxiolytics, approved mood stabilizers, Hypotensive agents-such as alpha or beta blockers, and ACE Inhibitors, Arrythmia agents, Warfarin or other anti-coagulants, digoxin, cyclosporine, rifampin, or MAO Inhibitors. Additionally they may not be otherwise involved in a clinical trial.

Eligible subjects are evaluated for safety paramaters prior to and throughout the trial through a variety of methods including the Columbia-Suicide Severity Rating Scale (C-SSRS) (Posner, K et al, 2007); electrocardiogram; physical examination; vital signs and body weight; and clinical laboratory testing including a Complete Metabolic Panel (Na, K, Cl, CO2, Glu, BUN, Cr, Ca, TP, Alb, TBili, AP, AST, ALT), Hematology CBC (Hgb, Hct, RBC, WBC, Plt, Diff), TSH (w/reflex T-4), Serum hCG for all females and urine analysis.

Eligible subjects not eliminated by the safety parameters are randomized to receive carvedilol (6.5 to 15.625 mgs daily total dose by oral administration) or placebo for a tour of duty and are followed for six months following the end of the tour in double-blind treatment according to the following schedule:

TABLE 6
Treatment Protocol

| | | | | Daily Dose |
|---|---|---|---|---|
| Week | Phase | AM | PM | Range |
| 0 | Titration Phase | 1 tab | 1 tab | 2 tablets |
| 1 | Treatment | 1-2 tabs | 1-2 tabs | 2-4 tablets |
| | Phase | | | |
| 2 | Dose Challenge | 1-2 tabs | 1-3 tabs | 2-5 tablets |
| Continuing For | Stable Dose | 1-2 tabs | 1-3 tabs | 2-5 tablets |
| Term Of | | | | |
| Treatment | | | | |
| Last Week of | Taper Phase | 1 tab | 1 tab | 2 tablets |
| Treatment | | | | |

Subjects will be treated while actively engaged in potential exposure to traumatic events and followed for six months following the end of exposure. Visits and evaluations are preformed according to the following schedule of events:

TABLE 7
Schedule of Events

| | | | | | | | Continuing | Visit 7/ | Visit 8/ | |
|---|---|---|---|---|---|---|---|---|---|---|
| | | | | | | | Protocol for | Wk 5; | Follow-up | |
| | | | | | | | weeks of term | Continued | post | Follow-up |
| | Visit 1/ | Visit 2/ | Visit 3/ | Visit 4/ | Visit 5/ | Visit 6/ | through | ever 7^{th} | termination | Phone |
| Procedure | Screen | Baseline | Week 1 | Week 2 | Week 3 | Week 4 | follow up | visit or ET | of study | call |
| Day | −3 to −28 | 0 | 7 +/− 3 | 14 +/− 3 | 21 +/− 3 | 28 +/− 3 | | 35 +/− 3 | 42 +/− 3 | 49 |
| Informed Consent | X | | | | | | | | | |
| Patient | X | | | | | | | | | |
| Demographics | | | | | | | | | | |
| Vital Signs | X | X | X | X | X | X | X | X | X | |
| Height | X | | | | | | | | | |
| Weight | X | X | X | X | X | X | X | X | X | |
| 12-lead ECG | X | | | | | | | X | | |
| Physical | X | | | | | | | X | | |
| Examination | | | | | | | | | | |
| Medical History | X | | | | | | | | | |
| Psychiatric History | X | | | | | | | | | |
| Diagnosis | X | | | | | | | | | |
| Inclusion/Exclusion | X | X | | | | | | | | |
| Criteria | | | | | | | | | | |
| Lab Work | X | | | | | | | X | | |
| Urine Drug Screen | X | | | | | | | | | |
| Serum Pregnancy | X | | | | | | | X | | |
| (females only) | | | | | | | | | | |
| MINI (version 6.0, | X | | | | | | | | | |
| January 2009) | | | | | | | | | | |
| CAPS (Westhers, F D) | X | | | | | | | X | | |
| et al, 1999) | | | | | | | | | | |
| DTS (Davidson, J R | X | X | X | X | X | X | X | X | | |
| et al, 1999) | | | | | | | | | | |
| ISI (Bastien, C H et | X | X | X | X | X | X | X | X | | |
| al, 2001) | | | | | | | | | | |
| C-SSRS (Posner, K | X | | | | | | | X | | |
| et al, 2007) | | | | | | | | | | |
| CGI (Guy, 1976) | X | X | X | X | X | X | X | X | | |
| Adverse Events | | | X | X | X | X | | X | X | X |
| Medication | | X | X | X | X | X | | X | | |
| Dispensed | | | | | | | | | | |

At visit 7 and every 7^{th} visit thereafter, Clinical Laboratory Testing (Week 5 or ET) include Complete Metabolic Panel (Na, K, Cl, CO2, Glu, BUN, Cr, Ca, TP, Alb, TBili, AP, AST, ALT), Hematology CBC (Hgb, Hct, RBC, WBC, Plt, Diff), and Serum hCG for all females.

Efficacy is determined by measuring the change from baseline in the Davidson Trauma Scale total score at week 5 in comparison to placebo. Secondary efficacy variables will be the Clinician Administered PTSD Scale (CAPS). Insomnia Severity Index (ISI) and Clinical Global Impression (CGI) changes at week 5. Safety measures will be collected and evaluated through the trial, specifically measuring changes from baseline visit to week 5. These measures include adverse event reports, physical examinations, vital signs, weight measurements, ECGs, clinical laboratory test results, and vital signs as well as scores for suicidal behaviors and/or ideation. Adverse events are any untoward medical event occurring in a subject administered study drug, irrespective of whether it has a causal relationship to the study drug. An adverse event can therefore be any unfavorable or unintended sign (including abnormal laboratory findings, for example), symptom, or disorder temporarily associated with study drug, whether or not considered related to the study drug.

Patients are allowed to take non-benzodiazepine sleep agents throughout the study including Zolpidem, Zaleplon and Eszopidone. They may also use lorazepam at a dose of no more than 2 mg/day for three days/week as a rescue medication if necessary.

Subjects are considered to have completed the study if they complete all of the visits. They may be terminated from the study if they fail to meet inclusion/exclusion criteria; suffer from an adverse event, have an insufficient therapeutic response, withdraw their consent, violate the protocol, stop coming, or die.

### Example 6

### Measurement of Pharmacokinetic Properties of Carvedilol Administered by Oral Dosage Form and Transdermal Patch

The pharmacokinetic properties of orally and transdermal patch delivered carvedilol can be measured in a human subject or animal model using standard procedures. In brief, a carvedilol oral dosage form of between 5-15 mg can be administered to a human subject in a tablet or capsule. A similar dosage can be administered to a human subject from a transdermal patch that is formulated and evaluated for skin permeation (such as as described by Ubaidulla et al., 2007). After administration of carvedilol, blood samples are drawn from the subject at time intervals (such as 1, 2, 3, 5, 8, 12, and 24 hours), plasma is separated by centrifugation, and plasma samples are stored at −70° C. until analyzed. The plasma carvedilol concentration is measured by reverse phase-high performance liquid chromatography (Ubaidulla et al., 2007).

The plasma concentration of carvedilol at different time intervals is subjected to pharmacokinetic analysis to calculate various parameters: maximum plasma concentration (Cmax), time to reach maximum concentration (Tmax), and area under the plasma concentration-time curve (AUC_{0→24}) as described (Ubaidulla et al., 2007). The values of Cmax and Tmax are read directly from the arithmetic plot of time vs plasma concentration of carvedilol. The AUC is calculated by using the trapezoidal rule. The elimination rate constant (Ke) is calculated by regression analysis from the slope of the line, and the half-life (t½) is obtained by 0.693/Ke. The relative bioavailability of the carvedilol after the transdermal administration versus the oral administration is calculated as follows: F(%)=(Sample AUC/Oral AUC) (Oral/Sample). The statistical significance of the differences between formulations is analyzed by Student t test using Graph Pad InStat 3 software. A difference below the probability level of 0.05 is considered statistically significant.

Following this procedure, an approximately similar Cₘₐₓ (μg/ml) is obtained for oral and transdermal patch administration of carvedilol, in which the Cₘₐₓ for the oral administration is obtained at 2 hours, whereas the Cₘₐₓ for the transdermal administration is obtained at 12 hours. The AUC (μg/ml) obtained for the transdermal patch administered carvedilol is approximately 2.5 to 3 fold greater than that obtained for oral administration.

### Example 7

### Phosphorylation of Carvedilol and Dephosphorylation by Alkaline Phosphatase

Carvedilol has low aqueous water solubility and absorption, and is categorized as a Class 4 drug substance (low solubility and low absorption) according to the criteria in the Biopharmaceutics Classification System, prepared by the Center for Drug Evaluation and Research (CDER) at the U.S. Food and Drug Administration (FDA) (http://www.fda.gov/downloads/Drugs/GuidanceComplianceRegulatoryInformation/Guiciances/UCM070246.pdf). To increase the aqueous solubility and likely absorption of carvedilol, a phosphate ester containing carvedilol was generated. Phosphate esters have been effective at increasing the water solubility of poorly soluble drugs, such as fospropofol and fosamprenaavir.

A phopho ester disodium salt of carvedilol, as shown in FIG. 4, was generated by dissolving the free base of carvedilol in dimethylformamide (DMF) and adding a 3-fold molar equivalent of phosphorus oxychloride (POCl₃). Specifically, carvedilol (124 mg, 0.311 mmol) was dissolved in 10 mL of dimethylformamide (DMF) to which 83 μL (0.93 mmol) of POCl₃ was added. Upon addition of the POCl₃, the solution became yellow. The tube was capped and allowed to sit overnight. A 10 μL aliquot was diluted to 1 mL with acetonitrile and analyzed by HPLC, using a Agilent 1260 Infinity HPLC system run with a Zorbax C-18 column, 69:31 acetonitrile (ACN):Carvedilol Buffer (2.72 g/L of monobasis potassium phosphate, pH 2.0), 20 μL loop, 55° C., 1.0 mL/min, UV detected at 240 nm. The starting carvedilol material had a retention time of about 16.6 minutes, whereas the DMF reaction material had a retention time of about 2 minutes, consistent with the formation of the carvedilol phopho ester disodium salt.

The DMF reaction mixture was purified by solid phase extraction (SPE), using a BondElut C-18 SPE cartridge, 100 mg, 3 mL (Agilent). The SPE was performed by first passing 1 mL of methanol over the column, followed by passing 1 mL of the mobile phase (69:31 CAN:Carvedilol Buffer). The sample (100 μL of 43.5 mM phosphocarvedilol in 1 mL mobile phase) was loaded on the column at a rate no faster than 5.0 mL/minute. The column was washed with 2 mL H₂O, and then eluted with 1 mL methanol. The sample was then evaporated to dryness.

The dried residue was reconstituted in a 1 mL solution of TRIS (pH 8.1, 0.05M) and NaCl (0.1M) for dephosphorylation with alkaline phosphatase. Dephosphorylation was initiated by adding 10 μL (1390 Units) of alkaline phosphatase (Sigma P0114) and incubating overnight at 37° C. 10 μL aliquots were removed and diluted to 100 μL in mobile phase before analyzing by injecting onto the HPLC column (as described above) in 20 μL amounts. After treatment with the alkaline phosphatase, a carvedilol peak with a retention time of about 14.95 minutes was detected.

In summary, the carvedilol peak completely disappeared following phosphorylation, as determined by HPLC, consistent with the formation of phosphorylated carvedilol. When the isolated phosphorylated carvedilol was reacted with alkaline phosphatase, the carvedilol HPLC peak reappeared. The generation of carvedilol by enzymatic dephosphorylation of the DMF reaction product provides evidence that the carvedilol was indeed phosphorylated.

All publications and patents cited herein are incorporated herein by reference for the purpose of describing and disclosing, for example, the materials and methodologies that are described in the publications, which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### REFERENCES

American Psychiatric Association: Diagnostic and Statistical Manual of Mental Disorders, Fourth Edition, Text Revision. Washington, D.C., 2000.
American Psychiatric Association: Diagnostic and Statistical Manual of Mental Disorders, Third Edition. Washington, D.C., 1980.
Bastien, C H, A Vallieres, and C M Morin. Validation of the Insomnia Severity Index as an Outcome Measure for Insomnia Research. Sleep Medicine. July 2001. Vol. 2. Iss. 4. Pp. 297-307.
Beck A T (2006). Depression: Causes and Treatment. Philadelphia: University of Pennsylvania Press. ISBN 0-8122-1032-8.
Beck, A. T., R. A Steer (1991) Manual for Beck Scale For Suicide Ideation. San Antonio, Tex.: Psychological Corporation.
Beck, A. T., R. A Steer (1988) Manual for Beck Hopelessness Scale. Psychological Corp., Harcourt Brace Jovanovich San Antonio, Tex.
Blake, D. D., Weathers, F. W., Nagy, L. M., Kaloupek, D. G., Gusman, F. D., Charney, D. S., & Keane, T. M. (1995). The development of a clinician-administered PTSD scale. Journal of Traumatic Stress, 8, 75-90.
Brennan, Francis et al. Stress-induced increases in avoidance responding: an animal model of post-traumatic stress disorder behavior? Neuropsychiatr Dis Treat. 2005 March; 1(1): 69-72.
Brady, K, T Pearlstein, M G Asnis et al. Efficacy and Safety of Sertraline Treatment of Post-traumatic Stress Disorder: A Randomized Controlled Trial. JAMA. 2000. Vp;/283(14). Pp. 1837-1844.
Coric, Vladimir•Stock, Elyse G•Pultz, Joseph•Marcus, Ronald•Sheehan, David V. Sheehan Suicidality Tracking Scale (Sheehan-STS): Preliminary Results from a Multicenter Clinical Trial in Generalized Anxiety Disorder. Psychiatry (Edgmont (Pa.: Township)) 2009 6 (1): 26-31.
Davidson, J. R. T., Kudler, H. S., & Smith, R. D. (1990). Assessment and pharmacotherapy of post-traumatic stress disorder. In J. E. L. Giller (Ed.), Biological assessment and treatment of post-traumatic stress disorder (pp. 205-221). Washington, D.C.: American Psychiatric Press.
Davidson J R, Weisler R H, Malik M L, Connor M K. Treatment of post-traumatic stress disorder with nefazodone. International Clinical Psychopharmacology 1998; 13(3):111-3.
Davidson, J. R. T., Book, S. W., Colket, J. T., Tupler, L. A., Roth, S., Hertzberg, M., Mellman, T., Beckham, J. C., Smith, R. D., Davidson, R. M., Katz, R., & Feldman, M. E. Assessment of a new self-rating scale for post-traumatic stress disorder. Psychological Medicine. 1999. Vol. 27. Pp. 153-160.
Davidson, J R, B O Rothbaum, B A van der Kolk, C R Sikes and G M Farfel. Multicenter. Double-Blind Comparison of Sertraline and Placebo in the Treatment of Post-traumatic Stress Disorder. Archives of General Psychiatry. 2001. Vol 58. PP. 485-492.
Foa, E & Tolin, D F (2006). Comparison of the PTSD Symptom Scale-Interview Version and the Clinician Administered PTSD Scale. Journal of Traumatic Stress, 13, 181-191.
Friedman M J, Marmar C R, Baker D G, Sikes C R, Farfel G M. Randomized, double-blind comparison of sertraline and placebo for posttraumatic stress disorder in a Department of Veterans Affairs setting. J Clin Psychiatry 2007:68:711-20.
Goodman W K, Price L H, Rasmussen S A, et al.: The Yale-Brown Obsessive Compulsive Scale, I: development, use, and reliability. Archives of General Psychiatry 46:1006-1011, 1989a.
Guy W. Clinician Global Impression (CGI). ECDEU Assessment Manual for Psychopharmacology. 1976. Rockville, Md., U.S. Department of Health, Education, and Welfare.
Hamilton, M. (1959). The assessment of anxiety states by rating. British Journal of Medical Psychology, 32, 50-55.
Hamilton M. A rating scale for depression. J. Neurol. Neurosurg. Psychiat., 1960, 23, 56.
Holbrook, T L, M R Galarneau, J L Dye, K Quinn and A L Dougherty. Morphine Use After Combat Injury in Iraq and Post-Traumatic Stress Disorder. January 2010. New England Journal of Medicine. Vol 362:2 Pp. 110-117.
Horowitz, M. J., Wilner, N. R., & Alvarez, W. (1979). Impact of Event Scale. A measure of subjective stress. Psychosomatic Medicine, 41, 209-218.
Kay S R, Fiszbein A, Opler L A: The Positive and Negative Syndrome Scale (PANSS) for schizophrenia. Schizophrenia Bulletin 13:261-276, 1987a.
Kessler, R C et al. Postraumatic stress disorder in the national Comorbity Survey. Arch Gen Psychiatry, 1995; 52. 1048-1060.
Kessler, Ronald C., Wai Tat Chiu, Olga Demler. Ellen E. Walters. Prevalence, Severity, and Comorbidity of 12-Month DSM-IV Disorders in the National Comorbidity Survey Replication 2005 Arch Gen Psychiatry. 62:617-627.
Khan, A, R L Kolts, M H Rapaport, K R R Krishanan, A E Brodhead and W A Brown. Magnitude of Placebo Response and Drug-Placebo Differences Across Psychiatric Disorders. Psychological Medicine. 2005. Vol. 35. Pp 743-749.
Krystal J H, Rosenheck R A, Cramer J A, Vessicchio J C. Jones K M, Vertrees J E, Horney R A, Huang G D, Stock C. Adjunctive risperidone treatment for antidepressant-resistant symptoms of chronic military service-related PTSD: a randomized trial. Jama 2011; 306:493-502.
Kovacs M. Rating scales to assess depression in school-aged children. Acta Paedopsychiatr. 1981; 46:305-315.
Liebowitz M R. Lebowitz Social Anxiety Scale Social Phobia. Mod Probl Pharmacopsychiatry 1987; 22:141-173
Levy, Aharon. An animal model for studying therapeutic drugs against post-traumatic stress disorder; Military Medicine, December 2001.
Maier S F. Exposure to the stressor environment prevents the temporal dissipation of behavioral depression/learned helplessness. Biol Psychiatry. 2001; 49:763-73.
Marshall, R D, K L Beebe, M Oldham and R Zarinelli. Efficacy and Safety of Paroxetine Treatment for Chronic PTSD: A Fixed-Dose, Placebo-Controlled Study. American Journal of Psychiatry. 2001. Vol. 158. Pp. 1982-1988.
Montgomery, S. A. & Åsberg, M. (1979) A New Depression Scale Designed To Be Sensitive To Change. British Journal of Psychiatry. Vol. 134. pp. 382-389.
Muigg, Patrik, Alfred Hetzenauer, Gabriele Hauer, Markus Hauschild, Stefano Gaburro, Elisabeth Frank, Rainer Landgraf, and Nicolas Singewald Impaired extinction of learned fear in rats selectively bred for high anxiety—evidence of altered neuronal processing in prefrontal-amygdala pathways Eur J Neurosci. 2008 December; 28(11): 2299-2309.
Nader, K., Kriegler, J. A., Blake, D. D., Pynoos, R. S., Newman, E., Weathers, F. W. (1996). Clinician Administered PTSD Scale, Child and Adolescent Version. White River Junction, Vt.: National Center for PTSD.
Olsen, L. R., et al., The internal and external validity of the Major Depression Inventory in measuring severity of depressive states Psychological Medicine (2003), 33:351-356 Cambridge University Press.
Pitman, R K, K M Sanders, R N Zusman, A R Healy. F Cheema, et al. Pilot Study of Secondary preention of Post-traumatic Stress Disorder with Propranolol. Society of Biological Psychiatry. 2002. Vol. 51. Pp. 189-192.
Posner K, Oquendo M, Gould M, et al. Columbia Classification Algorithm of Suicide Assessment (C CASA): classification of suicidal events in the FDA's pediatric suicidal risk analysis of antidepressants. Am J Psychiatry. 2007; 165:1035-1043.
Sheehan D V, Y Lecrubier, K H Sheehan, P Amorim, J Janavs, E Weiller, T Hergueta, R Baker, G C Dunbar. The Mini-International Neuropsychiatric Interview (M.I.N.I.): the development and validation of a structured diagnostic psychiatric interview for DSM-IV and ICD-10. J Clin Psychiatry. 1998; 59 Suppl. 20:2:2-33; quiz 34-57.
Southwick, S M, J H Krystal, J D Bremner, C A Morgan 3^{rd}, et al. Noradrenergic and Serotonergic Function in Post-traumatic Stress Disorder. Archives of General Psychiatry. 1997. Vol. 54. Iss. 8. Pp. 749-758.
Tucker, P, R Zaninelli, R Yehuda, L Ruggiero, K Dillingham and C D Pitts. Paroxetine in the Treatment of Chronic Post-traumatic Stress Disorder: Results of a Placebo-Controlled, Flexible-Dosage Trial. Journal of Clinical Psychiatry. 2001. Vol. 62(11). Pp. 860-868.
Ubaidulla, U, M V S Reddy, K Ruckmani, F J Ahmad, and R K Khar. Transdermal Therapeutic System of Carvedilol: Effect of Hydrophilic and hydrophobic Matrix on In vitro and In vivo Characteristics. AAPS PharmSciTech 2007; 8(1) E1-8.
Warren, W. L. Revised Hamilton Rating Scale for Depression (RHSD). (1994) Los Angeles, Western Psychological Services.
Watson C G, Juba M P, Manifold V, Kucala T, Anderson P E. The PTSD interview: rationale, description, reliability, and concurrent validity of a DSM-III-based technique. J. Clin Psychol. 1991 March; 47(2):179-88.
Weathers, F W, D D Blake. K E Krinsley, W Haddad, A M Ruscio, T M Keane, et al. Reliability and Validity of the Clinician Administered PTSD Scale. 1999. Auburn, Ala.: Auburn University. Department of Psychology.
Weiss, D. S., & Marmar, C. R. (1996). The Impact of Event Scale—Revised. In J. Wilson & T. M. Keane (Eds.), Assessing psychological trauma and PTSD (pp. 399-411). New York: Guilford.
Young R C, Biggs J T, Ziegler V E, Meyer D A. A rating scale for mania: reliability, validity and sensitivity. Br J Psychiatry. 1978; 133:429-435.
Zhe, Du et al. Expressions of Hippocampal Mineralocorticoid Receptor (MR) and Glucocorticoid Receptor (GR) in the Single-Prolonged Stress-Rats Acta Histochem Cytochem. 2008 Aug. 28; 41(4): 89-95.
Zoladz, Philip, Cheryl D. Conrad, Monika Fleshner, and David M. Diamond. Acute episodes of predator exposure in conjunction with chronic social instability as an animal model of post-traumatic stress disorder. Stress. 2008; 11(4): 259-281.

What is claimed is:

1. A method for preventing, ameliorating or alleviating symptoms of an anxiety disorder in a human subject suffering from or at risk for the anxiety disorder comprising administering to a human in need of such treatment a composition comprising an α blocker and a β blocker in an amount effective to prevent, ameliorate or alleviate one or more symptoms of said anxiety disorder.

2. The method of claim 1, wherein the anxiety disorder is Post Traumatic Stress Disorder (PTSD).

3. The method of claim 2, wherein the α and β blocker are administered prior to a traumatic event.

4. The method of claim 2, wherein the α and β blocker are administered after a traumatic event.

5. The method of claim 2, wherein the α and β blocker are administered after development of symptoms of PTSD.

6. The method of claim 1, wherein the symptom is panic, persistent worry, doubt, dread, fear, uneasiness, obsessive thoughts, repeated thoughts, flashbacks of traumatic experiences, mood instability, agitation, restlessness, dyspepsia, headaches, dyspnea, nightmares, ritualistic behaviors, insomnia, cold or sweaty hands and/or feet, shortness of breath, palpitations, hyper alertness, exaggerated startle response, avoidance of particular activities, avoidance of particular thoughts, diminished intensity of feelings, dry mouth, numbness or tingling in the hands or feet, nausea, muscle tension, or dizziness.

7. The method of claim 1, wherein the α blocker and β blocker are different compounds.

8. The method of claim 7, wherein the α blocker is doxazosin, silodosin, prazosin, tamsulosin, alfuzosin, terazosin, trimazosin, phenoxybenzamine or phentolamine.

9. The method of claim 7 wherein the β blocker is alpreolol, bucindolol, carteolol, nadolol, penbutolol, pindolol, propanolol, timolol, acebutolol, atenolol, betaxolol, bisoprolol, celiprolol, esmolol, metoprolol, nebivolol, butazamine, ICI-118,551. or SR 59230A.

10. The method of claim 1, wherein the α blocker and β blocker are the same compound.

11. The method of claim 10, wherein the α and β blocker is carvedilol.

12. The method of claim 11, wherein the carvedilol is administered in a symptom reducing effective dosage of about 0.25 to about 100 mg per clay.

13. The method of claim 11, wherein the carvedilol is administered in a symptom reducing effective dosage of from about 3 to about 17 mg per day.

14. The method of claim 10, wherein the carvedilol is administered by a transdermal patch.

15. The method of claim 14, wherein the carvedilol is administered in a symptom reducing effective dosage of from about 3 to about 15 mg per day.

16. The method of claim 10, wherein the α and β blocker is labetalol.

17. A method for preventing, ameliorating, or alleviating symptoms of an anxiety disorder in a human subject suffering from or at risk for the anxiety disorder comprising coordinately administering a psychotherapeutic agent in an amount effective to prevent, ameliorate or alleviate said anxiety disorder, an α blocker, and a β blocker in an a amount effective to reduce, prevent or treat one or more symptoms of the anxiety disorder.

18. The method of claim 17, wherein the anxiety disorder is Post Traumatic Stress Disorder (PTSD).

19. The method of claim 18, wherein the coordinate administration is prior to a traumatic event.

20. The method of claim 18, wherein the coordinate administration is after a traumatic event.

21. The method of claim 18, wherein the coordinate administration is after development of symptoms of PTSD.

22. The method of claim 17, wherein the symptom is panic, persistent worry, doubt, dread, fear, uneasiness, obsessive thoughts, repeated thoughts, flashbacks of traumatic experiences, mood instability, agitation, restlessness, dyspepsia, headaches, dyspnea, nightmares, ritualistic behaviors, insomnia, cold or sweaty hands and/or feet, shortness of breath, palpitations, hyper alertness, exaggerated startle response, avoidance of particular activities, avoidance of particular thoughts, diminished intensity of feelings, dry mouth, numbness or tingling in the hands or feet, nausea, muscle tension, or dizziness.

23. The method of claim 17, wherein the α blocker and β blocker are different compounds.

24. The method of claim 23, wherein the α blocker is doxazosin, silodosin, prazosin, tamsulosin, alfuzosin, terazosin, trimazosin, phenoxybenzamine or phentolamine.

25. The method of claim 23 wherein the β blocker is alpreolol, bucindolol, carteolol, nadolol, penbutolol, pindolol, propanolol, timolol, acebutolol, atenolol, betaxolol, bisoprolol, celiprolol, esmolol, metoprolol, nebivolol, butazamine, ICI-118,551, or SR 59230A.

26. The method of claim 17, wherein the α blocker and β blocker are the same compound.

27. The method of claim 26, wherein the α and β blocker is carvedilol.

28. The method of claim 27, wherein the carvedilol is administered in a symptom reducing effective dosage of about 0.25 to about 100 mg per day.

29. The method of claim 27, wherein the carvedilol is administered in a symptom reducing effective dosage of from about 3 to about 17 mg per day.

30. The method of claim 26, wherein the carvedilol is administered by a transdermal patch.

31. The method of claim 30, wherein the carvedilol is administered in a symptom reducing effective dosage of from about 3 to about 15 mg per day.

32. The method of claim 26, wherein the α and β blocker is labetalol.

33. The method of claim 17, wherein the psychotherapeutic agent is an anti-depressant drug.

34. The method of claim 33, wherein the anti-depressant drug is tri-cyclic anti-depressants (TCAs), specific monoamine reuptake inhibitors, selective serotonin reuptake inhibitors (SSRIs), selective norepinephrine reuptake inhibitors, selective dopamine reuptake inhibitors, multiple monoamine reuptake inhibitors, monoamine oxidase inhibitors (MAOIs), or indeterminate (atypical) anti-depressants.

35. The method of claim 34, wherein the SSRI is citalopram.

36. The method of claim 17, wherein the psychotherapeutic agent is an anti-convulsant.

37. The method of claim 36, wherein the anti-convulsant is lamotrigine, carbamazepine, oxcarbazepine, valproate, levetriacetam, or topiramate.

38. The method of claim 17, wherein the psychotherapeutic agent is administered by a mode of delivery selected from oral, buccal, nasal, aerosol, topical, transdermal, intramuscular, mucosal, or injectable delivery.

39. The method of claim 17, wherein the psychotherapeutic agent is administered in a sustained release formulation.

40. The method of claim 30, wherein the sustained release formulation is a sustained release, oral, transdermal, or injectable formulation.

41. The method of claim 39, wherein the psychotherapeutic agent is administered in a dosage of from about 60 mg to about 1000 mg.

42. The method of claim 17, wherein the psychotherapeutic agent is an anxiolytic drug.

43. The method of claim 17, wherein the psychotherapeutic agent and the α and β blocker are administered to said subject simultaneously.

44. The method of claim 17, wherein psychotherapeutic agent and the α and β blocker are administered in a single, combined dosage form.

45. The method of claim 17, wherein the psychotherapeutic agent, the α blocker, and the β blocker are administered to said subject at different times during a coordinate dosing period.

46. A pharmaceutical composition for preventing, ameliorating, or alleviating symptoms of an anxiety disorder in a human subject suffering from or at risk for the anxiety disorder comprising a psychotherapeutic agent in an amount effective to treat said anxiety disorder, an α blocker and a β blocker in an amount effective to reduce symptoms in said subject, wherein said psychotherapeutic agent and the α and β blockers are admixed or co-formulated in a single, combined dosage form.

47. A pharmaceutical composition according to claim 46, wherein said anxiety disorder is PTSD.

48. The pharmaceutical composition according to claim 46, wherein said psychotherapeutic therapeutic agent is selected from anti-depressant, anxiolytic, anticonvulsant, antipsychotic, antiaddictive, appetite suppressant drugs and opiate agonists.

49. The pharmaceutical composition according to claim 48, wherein the anti-depressant drug is selected from tri-cyclic anti-depressants (TCAs), specific monoamine reuptake inhibitors, selective serotonin reuptake inhibitors (SSRIs), selective norepinephrine reuptake inhibitors, selective dopamine reuptake inhibitors, multiple monoamine reuptake inhibitors, monoamine oxidase inhibitors (MAOIs), and indeterminate (atypical) anti-depressants.

50. The pharmaceutical composition according to claim 49, wherein the SSRI is citalopram.

51. The pharmaceutical composition according to claim 46, wherein the α blocker and β blocker are different compounds.

52. The pharmaceutical composition according to claim 51, wherein the α blocker is doxazosin, silodosin, prazosin, tamsulosin, alfuzosin, terazosin, trimazosin, phenoxybenzamine or phentolamine.

53. The pharmaceutical composition according to claim 41, wherein the β blocker is alpreolol, bucindolol, carteolol, nadolol, penbutolol, pindolol, propanolol, timolol, acebutolol, atenolol, betaxolol, bisoprolol, celiprolol, esmolol, metoprolol, nebivolol, butazamine, ICI-118,551, or SR 59230A.

54. The pharmaceutical composition according to claim 46, wherein the α blocker and β blocker are the same compound.

55. The pharmaceutical composition according to claim 54, wherein the α and β blocker is carvedilol.

56. The pharmaceutical composition according to claim 55, wherein the carvedilol is in a symptom reducing effective dosage of about 0.25 to about 100 mg per day.

57. The pharmaceutical composition according to claim 55, wherein the carvedilol is in a symptom reducing effective dosage of from about 3 to about 17 mg per day.

58. The pharmaceutical composition according to claim 55, wherein the carvedilol is formulated for administration by a transdermal patch mode of delivery.

59. The pharmaceutical composition according to claim 55, wherein the carvedilol is in a symptom reducing effective dosage of from about 3 to about 15 mg per day.

60. The pharmaceutical composition according to claim 54, wherein the α and β blocker is labetalol.

61. A pharmaceutical composition according to claim 46, wherein the psychotherapeutic agent is formulated for administration by a mode of delivery selected from selected from oral, buccal, nasal, aerosol, topical, transdermal, mucosal, or injectable delivery.

62. The pharmaceutical composition according to claim 46, wherein the psychotherapeutic agent is in a sustained release formulation.

63. The pharmaceutical composition according to claim 62, wherein the sustained release formulation provides therapeutically effective plasma levels of said psychotherapeutic drug over a sustained delivery period of approximately 8 hours or longer.

64. The pharmaceutical composition according to claim 62, wherein the sustained release formulation provides therapeutically effective plasma levels of said psychotherapeutic agent over a sustained delivery period of approximately 18 hours or longer.

65. A pharmaceutical composition according to claim 46, wherein the psychotherapeutic agent is administered in a dosage of from about 60 mg to about 1000 mg.

66. A method for preventing symptoms of an anxiety disorder in a human subject suffering from or at risk for the anxiety disorder comprising coordinately administering a psychotherapeutic agent in an amount effective to prevent, ameliorate or alleviate said anxiety disorder, and an α blocker in an a amount effective to prevent one or more symptoms of the anxiety disorder.

67. The method of claim 66, wherein the anxiety disorder is Post Traumatic Stress Disorder (PTSD).

68. The method of claim 67, wherein the coordinate administration is prior to a traumatic event.

69. The method of claim 66, wherein the symptom is panic, persistent worry, doubt, dread, fear, uneasiness, obsessive thoughts, repeated thoughts, flashbacks of traumatic experiences, mood instability, agitation, restlessness, dyspepsia, headaches, dyspnea, nightmares, ritualistic behaviors, insomnia, cold or sweaty hands and/or feet, shortness of breath, palpitations, hyper alertness, exaggerated startle response, avoidance of particular activities, avoidance of particular thoughts, diminished intensity of feelings, dry mouth, numbness or tingling in the hands or feet, nausea, muscle tension, or dizziness.

70. The method of claim 66, wherein the α blocker is doxazosin, silodosin, prazosin, tamsulosin, alfuzosin, terazosin, trimazosin, phenoxybenzamine or phentolamine.

71. The method of claim 66, wherein the psychotherapeutic agent is an anti-depressant, anxiolytic, anticonvulsant, antipsychotic, antiaddictive, appetite suppressant drug or opiate agonist.

72. The method of claim 71, wherein the anti-depressant drug is selected from tri-cyclic anti-depressants (TCAs), specific monoamine reuptake inhibitors, selective serotonin reuptake inhibitors (SSRIs), selective norepinephrine reuptake inhibitors, selective dopamine reuptake inhibitors, multiple monoamine reuptake inhibitors, monoamine oxidase inhibitors (MAOIs), and indeterminate (atypical) anti-depressants.

73. The method of claim 72, wherein the SSRI is citalopram.

74. The method of claim 71, wherein the psychotherapeutic agent is an anti-convulsant.

75. The method of claim 74, wherein the anti-convulsant is lamotrigine, carbamazepine, oxcarbazepine, valproate, levetriacetam, or topiramate.

76. The method of claim 66, wherein the psychotherapeutic agent is administered by a mode of delivery selected from oral, buccal, nasal, aerosol, topical, transdermal, intramuscular, mucosal, or injectable delivery.

77. The method of claim 66, wherein the psychotherapeutic agent is administered in a sustained release formulation.

78. The method of claim 77, wherein the sustained release formulation is a sustained release, oral, transdermal, or injectable formulation.

79. The method of claim 77, wherein the psychotherapeutic agent is administered in a dosage of from about 60 mg to about 1000 mg.

80. The method of claim 71, wherein the psychotherapeutic agent is an anxiolytic drug.

81. The method of claim 66, wherein psychotherapeutic agent and the α blocker are administered in a single, combined dosage form.

82. The method of claim 66, wherein the psychotherapeutic agent and the α blocker are administered to said subject at different times during a coordinate dosing period.

83. A method for preventing symptoms of an anxiety disorder in a human subject suffering from or at risk for the anxiety disorder comprising coordinately administering a psychotherapeutic agent in an amount effective to prevent, ameliorate or alleviate said anxiety disorder, and a β blocker in an a amount effective to prevent one or more symptoms of the anxiety disorder.

84. The method of claim 83, wherein the anxiety disorder is Post Traumatic Stress Disorder (PTSD).

85. The method of claim 83, wherein the coordinate administration is prior to a traumatic event.

86. The method of claim 83, wherein the symptom is panic, persistent worry, doubt, dread, fear, uneasiness, obsessive thoughts, repeated thoughts, flashbacks of traumatic experiences, mood instability, agitation, restlessness, dyspepsia, headaches, dyspnea, nightmares, ritualistic behaviors, insomnia, cold or sweaty hands and/or feet, shortness of breath, palpitations, hyper alertness, exaggerated startle response, avoidance of particular activities, avoidance of particular thoughts, diminished intensity of feelings, dry mouth, numbness or tingling in the hands or feet, nausea, muscle tension, or dizziness.

88. The method of claim 83, wherein β blocker is alpreolol, bucindolol, carteolol, nadolol, penbutolol, pindolol, propanolol, timolol, acebutolol, atenolol, betaxolol, bisoprolol, celiprolol, esmolol, metoprolol, nebivolol, butazamine, ICI-118,551, or SR 59230A.

89. The method of claim 83, wherein the psychotherapeutic agent is an anti-depressant, anxiolytic, anticonvulsant, antipsychotic, antiaddictive, appetite suppressant drug and opiate agonist.

90. The method of claim 89, wherein the anti-depressant drug is selected from tri-cyclic anti-depressants (TCAs), specific monoamine reuptake inhibitors, selective serotonin reuptake inhibitors (SSRIs), selective norepinephrine reuptake inhibitors, selective dopamine reuptake inhibitors, multiple monoamine reuptake inhibitors, monoamine oxidase inhibitors (MAOIs), and indeterminate (atypical) anti-depressants.

91. The method of claim 90, wherein the SSRI is citalopram.

92. The method of claim 89, wherein the psychotherapeutic agent is an anti-convulsant.

93. The method of claim 92, wherein the anti-convulsant is lamotrigine, carbamazepine, oxcarbazepine, valproate, levetriacetam, or topiramate.

94. The method of claim 83, wherein the psychotherapeutic agent is administered by a mode of delivery selected from oral, buccal, nasal, aerosol, topical, transdermal, intramuscular, mucosal, or injectable delivery.

95. The method of claim 83, wherein the psychotherapeutic agent is administered in a sustained release formulation.

96. The method of claim 95, wherein the sustained release formulation is a sustained release, oral, transdermal, or injectable formulation.

97. The method of claim 95, wherein the psychotherapeutic agent is administered in a dosage of from about 60 mg to about 1000 mg.

98. The method of claim 89, wherein the psychotherapeutic agent is an anxiolytic drug.

99. The method of claim 83, wherein psychotherapeutic agent and the β blocker are administered in a single, combined dosage form.

100. The method of claim 83, wherein the psychotherapeutic agent and the β blocker are administered to said subject at different times during a coordinate dosing period.

101. A method for preventing, ameliorating or alleviating symptoms of an anxiety disorder in a female human subject suffering from or at risk for the anxiety disorder comprising administering to a female human in need of such treatment a composition comprising an α blocker and a β blocker in an amount effective to prevent, ameliorate or alleviate one or more symptoms of said anxiety disorder.

102. The method of claim 101, wherein the anxiety disorder is Post Traumatic Stress Disorder (PTSD).

104. The method of claim 102, wherein the α and β blocker are administered prior to a traumatic event.

105. The method of claim 102, wherein the α and β blocker are administered after a traumatic event.

106. The method of claim 102, wherein the α and β blocker are administered after development of symptoms of PTSD.

107. The method of claim 101, wherein the symptom is panic, persistent worry, doubt, dread, fear, uneasiness, obsessive thoughts, repeated thoughts, flashbacks of traumatic experiences, mood instability, agitation, restlessness, dyspepsia, headaches, dyspnea, nightmares, ritualistic behaviors, insomnia, cold or sweaty hands and/or feet, shortness of breath, palpitations, hyper alertness, exaggerated startle response, avoidance of particular activities, avoidance of particular thoughts, diminished intensity of feelings, dry mouth, numbness or tingling in the hands or feet, nausea, muscle tension, or dizziness.

108. The method of claim 101, wherein the α blocker and β blocker are different compounds.

109. The method of claim 108, wherein the α blocker is doxazocin, silodosin, prazosin, tamsulosin, alfuzosin, terazosin, trimazosin, phenoxybenzamine or phentolamine.

110. The method of claim 109, wherein the β blocker is alpreolol, bucindolol, carteolol, nadolol, penbutolol, pindolol, propanolol, timolol, acebutolol, atenolol, betaxolol, bisoprolol, celiprolol, esmolol, metoprolol, nebivolol, butazamine, ICI-118,551, or SR 59230A.

110. The method of claim 101, wherein the α blocker and β blocker are the same compound.

112. The method of claim 101, wherein the α and β blocker is carvedilol.

113. The method of claim 112, wherein the carvedilol is administered in a symptom reducing effective dosage of about 0.25 to about 100 mg per day.

114. The method of claim 112, wherein the carvedilol is administered in a symptom reducing effective dosage of from about 3 to about 17 mg per day.

115. The method of claim 112, wherein the carvedilol is administered by a transdermal patch.

116. The method of claim 112, wherein the carvedilol is administered in a symptom reducing effective dosage of from about 3 to about 15 mg per day.

117. The method of claim 112, wherein the α and β blocker is labetalol.

118. A method for preventing, ameliorating or alleviating symptoms of an anxiety disorder in a human subject suffering from or at risk for the anxiety disorder comprising administering to a human in need of such treatment a composition comprising an α blocker and a β blocker in an amount effective to prevent, ameliorate or alleviate one or more symptoms of said anxiety disorder, wherein the human is not a military veteran.

119. The method of claim 118, wherein the anxiety disorder is Post Traumatic Stress Disorder (PTSD).

120. The method of claim 119, wherein the α and β blocker are administered prior to a traumatic event.

121. The method of claim 119, wherein the α and β blocker are administered after a traumatic event.

122. The method of claim 119, wherein the α and β blocker are administered after development of symptoms of PTSD.

123. The method of claim 118, wherein the symptom is panic, persistent worry, doubt, dread, fear, uneasiness, obsessive thoughts, repeated thoughts, flashbacks of traumatic experiences, mood instability, agitation, restlessness, dyspepsia, headaches, dyspnea, nightmares, ritualistic behaviors, insomnia, cold or sweaty hands and/or feet, shortness of breath, palpitations, hyper alertness, exaggerated startle response, avoidance of particular activities, avoidance of particular thoughts, diminished intensity of feelings, dry mouth, numbness or tingling in the hands or feet, nausea, muscle tension, or dizziness.

124. The method of claim 118, wherein the α blocker and β blocker are different compounds.

125. The method of claim 124, wherein the α blocker is doxazosin, silodosin, prazosin, tamsulosin, alfuzosin, terazosin, trimazosin, phenoxybenzamine or phentolamine.

126. The method of claim 124, wherein the β blocker is alpreolol, bucindolol, carteolol, nadolol, penbutolol, pindolol, propanolol, timolol, acebutolol, atenolol, betaxolol, bisoprolol, celiprolol, esmolol, metoprolol, nebivolol, butazamine, ICI-118,551, or SR 59230A.

127. The method of claim 118, wherein the α blocker and β blocker are the same compound.

128. The method of claim 127, wherein the α and β blocker is carvedilol.

129. The method of claim 128, wherein the carvedilol is administered in a symptom reducing effective dosage of about 0.25 to about 100 mg per day.

130. The method of claim 128, wherein the carvedilol is administered in a symptom reducing effective dosage of from about 3 to about 17 mg per day.

131. The method of claim 128, wherein the carvedilol is administered by a transdermal patch.

132. The method of claim 131, wherein the carvedilol is administered in a symptom reducing effective dosage of from about 3 to about 15 mg per day.

133. The method of claim 127, wherein the α and β blocker is labetalol.

134. A method for preventing, ameliorating or alleviating symptoms of an anxiety disorder in a human subject suffering from or at risk for the anxiety disorder comprising administering to a human in need of such treatment a sustained release composition of carvedilol comprising an amount effective to prevent, ameliorate or alleviate one or more symptoms of said anxiety disorder, wherein the amount of sustained release carvedilol provides an increased bioavailability compared to that achieved by the same amount of an immediate release, oral carvedilol.

135. The method of claim 134, wherein the anxiety disorder is Post Traumatic Stress Disorder (PTSD).

136. The method of claim 134, wherein the increased bioavailability is an increased area under the curve (AUC) of carvedilol in the subject that is two to ten times or more higher.

137. The method of claim 134, wherein the increased bioavailability is an increased area under the curve (AUC) of carvedilol in the subject that is ten times higher.

138. The method of claim 134, wherein the increased bioavailability is an increased area under the curve (AUC) of carvedilol in the subject that is five times higher.

139. The method of claim 134, wherein the increased bioavailability is an increased area under the curve (AUC) of carvedilol in the subject that is two times higher.

141. The method of claim 134, wherein the carvedilol is administered in a symptom reducing effective dosage of from about 3 to about 17 mg per day.

140. The method of claim 134, wherein the sustained release carvedilol is administered by a transdermal patch.

141. The method of claim 140, wherein the carvedilol is administered in a symptom reducing effective dosage of from about 3 to about 15 mg per day.

142. A method for preventing, ameliorating or alleviating symptoms of an anxiety disorder in a human subject suffering from or at risk for the anxiety disorder comprising administering to a human in need of such treatment a sustained release dosage form of carvedilol comprising an amount effective to prevent, ameliorate or alleviate one or more symptoms of said anxiety disorder.

143. The method of claim 142, wherein the anxiety disorder is Post Traumatic Stress Disorder (PTSD).

144. The method of claim 142, wherein the sustained release dosage form is an extended release formulation.

145. The method of claim 144, wherein the extended release formulation contains carvedilol combined with a sustained release vehicle, matrix, binder, or coating material.

146. The method of claim 145, wherein the sustained release vehicle is a topical formulation, polymer, aerosol particle, microparticle, microcapsule, microsphere, mini-tablet, multi-component formulation, drug-releasing lipid, osmotic dosage form, or drug-releasing wax.

147. The method of claim 142, wherein the sustained release dosage form contains carvedilol that is delivered by an extended release device or spray.

148. The method of claim 147, wherein the extended release device is a topical device, implant, inhaler, nebulizer, or dry powder.

149. The method of claim 148, wherein the extended release device is topical device comprising a transdermal patch.

150. The method of claim 149, wherein the transdermal patch is a matrix-type transdermal patch.

151. The method of claim 150, wherein the transdermal patch contains carvedilol dispersed in an adhesive polymer.

152. The method of claim 151, wherein the transdermal patch further contains a plasticizer or tackifying agent.

153. The method of claim 151, wherein the transdermal patch further contains a skin-penetration enhancer.

154. The method of claim 149, wherein the transdermal patch will result in a Cₘₐₓ that is reached at about 10-12 hours.

155. The method of claim 149, wherein the transdermal patch will result in an increased bioavailability of carvedilol compared to oral administration of carvedilol.

156. The method of claim 155, wherein the increased bioavailability is a two to five-fold or greater increase in area under the curve (AUC) over 24 hours.

157. A method for preventing, ameliorating or alleviating symptoms of Post Traumatic Stress Disorder (PTSD) in a human subject suffering from or at risk for PTSD comprising administering to a human in need of such treatment a composition of carvedilol comprising an amount effective to prevent, ameliorate or alleviate one or more symptoms of said PTSD.

158. The method of claim 157, wherein the carvedilol is administered through a transdermal patch.

159. The method of claim 158, wherein the amount of carvedilol administered is from about 3 to about 15 mg per day.

160. The method of claim 157, wherein the human subject is a female.

161. The method of claim 157, wherein the human subject is not a military veteran.

162. A method for preventing, ameliorating or alleviating symptoms of an anxiety disorder in a human subject suffering from or at risk for the anxiety disorder comprising administering to a human in need of such treatment a prodrug dosage form of carvedilol comprising an amount effective to prevent, ameliorate or alleviate one or more symptoms of said anxiety disorder.

163. The method of claim 162, wherein the anxiety disorder is Post Traumatic Stress Disorder (PTSD).

164. The method of claim 162, wherein the prodrug dosage form has improved solubility compared to an oral dosage form of carvedilol.

165. The method of claim 162, wherein the prodrug dosage form has improved bioavailability compared to an oral dosage form of carvedilol.

166. The method of claim 161, wherein the prodrug dosage form is selected from an ester or amino acid prodrug of carvedilol.

167. The method of claim 166, wherein the prodrug dosage form is a phospho ester carvedilol.

168. The method of claim 166, wherein the prodrug dosage form is a phosphocholine ester carvedilol.

169. A composition of carvedilol comprising a carvedilol prodrug.

170. The composition of claim 169, wherein said carvedilol prodrug has improved solubility compared to an oral dosage form of carvedilol.

171. The composition of claim 169, wherein said carvedilol prodrug has improved bioavailability compared to an oral dosage form of carvedilol.

172. The composition of claim 169, wherein said carvedilol prodrug has improved solubility and bioavailability compared to an oral dosage form of carvedilol.

173. The composition of claim 169, wherein said carvedilol prodrug is selected from an ester or amino acid prodrug of carvedilol.

174. The composition of claim 169, wherein said carvedilol prodrug is a phospho ester carvedilol.

175. The composition of claim 169, wherein said carvedilol prodrug is a phosphocholine ester carvedilol.

176. A method for preventing, ameliorating or alleviating symptoms of an anxiety disorder in a human subject suffering from or at risk for the anxiety disorder comprising administering to a human in need of such treatment a composition of carvedilol comprising an amount effective to prevent, ameliorate or alleviate one or more symptoms of said anxiety disorder, wherein the composition of carvedilol is administered by mucosal, nasal, intranasal, or aerosol delivery.

177. The method of claim 176, wherein the composition of carvedilol is administered by mucosal delivery.

178. The method of claim 177, wherein the amount of carvedilol administered is from about 3 to about 15 mg per day.

179. The method of claim 176, wherein the composition of carvedilol is administered by nasal delivery.
